(19) United States
(12) Patent Application Publication
(10) Pub. No.: US 2026/0253685 A1
(43) Pub. Date: Aug. 27, 2026

(54) MANAGEMENT OF EHR DATA

(71) Applicant: Helix, Inc., San Mateo, CA (US)

(72) Inventors: Lisa McEwen, Victoria (CA); Lance Eighme, San Mateo, CA (US); Nicole Washington, Albany, CA (US); Simon White, Redwood City, CA (US); Anna Swigart, San Mateo, CA (US); Oliver Tucher, San Mateo, CA (US)

(21) Appl. No.: 19/060,686

(22) Filed: Feb. 22, 2025

(51) Int. Cl. G16H 10/60; G16H 15/00

(52) U.S. Cl. CPC G16H 10/60; G16H 15/00

(57) ABSTRACT

Apparatus and method for managing Electronic Health Record (EHR) data. In an embodiment, an apparatus is configured to receive a batch of EHR data from a health partner, perform structural conformance validation of the EHR data based on structural compliance criteria stored in memory, and accept the EHR data as structurally-compliant EHR data when in compliance with the structural compliance criteria. The apparatus is configured to perform data quality validation of the structurally-compliant EHR data based on data quality compliance criteria stored in memory, accept the structurally-compliant EHR data as compliant EHR data when in compliance with the data quality compliance criteria, and store the compliant EHR data in a production dataset.

### TECHNICAL FIELD

The following disclosure relates to the field of health informatics, and in particular, to management of information stored in Electronic Health Records (EHRs).

### BACKGROUND

Healthcare professionals, researchers, and/or analytical teams continually seek out rich datasets to better understand relationships between various health conditions for patients, demographics, genetics, etc. Having access to detailed health/healthcare datasets (sometimes referred to as production datasets) on a population level helps to enable research insights that have historically been unavailable. In order to build effective health datasets, many entities rely on contributions from multiple sources. Unfortunately, even when these sources use a common format for their data (e.g., the Observational Medical Outcomes Partnership (OMOP) Common Data Model (CDM) format), each source may use different arrangements of data, and each source may be subject to its own idiosyncrasies. This can result in non-uniform datasets, which hampers the ability to draw out key insights or perform research activities using the aggregated datasets.

### SUMMARY

Embodiments described herein provide an automated solution for gathering and/or managing information based on Electronic Health Records (EHRs). As a general overview, an apparatus referred to as a management server, is configured to acquire EHR data from a health partner. Before the EHR data is added to a production dataset, the management server is configured to perform an initial validation of the EHR data to determine whether the EHR data complies structurally with a data model, and to perform subsequent validation of the EHR data to verify the accuracy or quality of the content contained in the EHR data. When the EHR data passes validation, the management server is configured to add the EHR data to the production dataset, which may be used for further research, analysis, etc. One technical benefit is the veracity of the production dataset is improved by validating the EHR data that is added.

In an embodiment (also referred to as an aspect), an apparatus such as a management server described above, comprises a network interface configured to communicate over a communication network, and a processor and memory. The memory is configured to store structural compliance criteria and data quality compliance criteria. The processor is configured to execute an algorithm to, during an ingestion phase, receive a batch of EHR data from a health partner via the network interface, perform structural conformance validation of the EHR data based on the structural compliance criteria by comparing the EHR data to a data model defined in the structural compliance criteria, determine whether the EHR data is in compliance with the structural compliance criteria according to the structural conformance validation, reject the EHR data when not in compliance with the structural compliance criteria, and accept the EHR data as structurally-compliant EHR data when in compliance with the structural compliance criteria. The processor is configured to execute the algorithm to, after the ingestion phase, perform data quality validation of the structurally-compliant EHR data based on the data quality compliance criteria, determine whether the structurally-compliant EHR data is in compliance with the data quality compliance criteria according to the data quality validation, reject the structurally-compliant EHR data when not in compliance with the data quality compliance criteria, accept the structurally-compliant EHR data as compliant EHR data when in compliance with the data quality compliance criteria, and store the compliant EHR data in a production dataset.

In an embodiment, a method comprises, during an ingestion phase, receiving a batch of EHR data from a health partner via a communication network, performing structural conformance validation of the EHR data based on structural compliance criteria stored in memory by comparing the EHR data to a data model defined in the structural compliance criteria, determining whether the EHR data is in compliance with the structural compliance criteria according to the structural conformance validation, rejecting the EHR data when not in compliance with the structural compliance criteria, and accepting the EHR data as structurally-compliant EHR data when in compliance with the structural compliance criteria. The method comprises, after the ingestion phase, performing data quality validation of the structurally-compliant EHR data based on data quality compliance criteria stored in memory, determining whether the structurally-compliant EHR data is in compliance with the data quality compliance criteria according to the data quality validation, rejecting the structurally-compliant EHR data when not in compliance with the data quality compliance criteria, accepting the structurally-compliant EHR data as compliant EHR data when in compliance with the data quality compliance criteria, and storing the compliant EHR data in a production dataset.

Other embodiments may include computer readable media, other systems, or other methods as described below.

The above summary provides a basic understanding of some aspects of the specification. This summary is not an extensive overview of the specification. It is intended to neither identify key or critical elements of the specification nor delineate any scope particular embodiments of the specification, or any scope of the claims. Its sole purpose is to present some concepts of the specification in a simplified form as a prelude to the more detailed description that is presented later.

### DESCRIPTION OF THE DRAWINGS

Some embodiments of the present disclosure are now described, by way of example only, and with reference to the accompanying drawings. The same reference number represents the same element or the same type of element on all drawings.

FIGS. 1A-1B are block diagrams of a health data management architecture in illustrative embodiments.

FIG. 2 is a block diagram illustrating genetic testing in an illustrative embodiment.

FIG. 3 is a flow chart illustrating a method of genetic testing in an illustrative embodiment.

FIG. 4 is a block diagram of management server in an illustrative embodiment.

FIG. 5 is a flow chart illustrating a method of managing EHR data in an illustrative embodiment.

FIG. 6 illustrates an ingestion phase of EHR data into a data management service in an illustrative embodiment.

FIG. 7A illustrates EHR data in an illustrative embodiment.

FIG. 7B illustrates OMOP CDM format in an illustrative embodiment.

FIG. 8 is a block diagram illustrating structural conformance validation in an illustrative embodiment.

FIGS. 9A-9B illustrate a report in an illustrative embodiment.

FIG. 10A illustrates accepting of EHR data during an ingestion phase in an illustrative embodiment.

FIG. 10B illustrates rejecting of EHR data during an ingestion phase in an illustrative embodiment.

FIGS. 11A-11C are flow charts illustrating additional steps of the method in FIG. 5 for managing EHR data in illustrative embodiments.

FIG. 12 is a block diagram illustrating data quality validation in an illustrative embodiment.

FIG. 13 illustrates a table in an illustrative embodiment.

FIG. 14 illustrates a measurement table in an illustrative embodiment.

FIG. 15 illustrates a vocabulary table in an illustrative embodiment.

FIG. 16 illustrates a condition occurrence table in an illustrative embodiment.

FIG. 17 illustrates a specimen table in an illustrative embodiment.

FIG. 18 illustrates a report in an illustrative embodiment.

FIG. 19A illustrates accepting of compliant EHR data in a production dataset in an illustrative embodiment.

FIG. 19B illustrates rejecting of the structurally-compliant EHR data from a production dataset in an illustrative embodiment.

FIG. 20 is a diagram illustrating use of an ML system to process EHR data in an illustrative embodiment.

### DETAILED DESCRIPTION

The figures and the following description illustrate specific exemplary embodiments. It will thus be appreciated that those skilled in the art will be able to devise various arrangements that, although not explicitly described or shown herein, embody the principles of the embodiments and are included within the scope of the embodiments. Furthermore, any examples described herein are intended to aid in understanding the principles of the embodiments, and are to be construed as being without limitation to such specifically recited examples and conditions. As a result, the inventive concept(s) is not limited to the specific embodiments or examples described below, but by the claims and their equivalents.

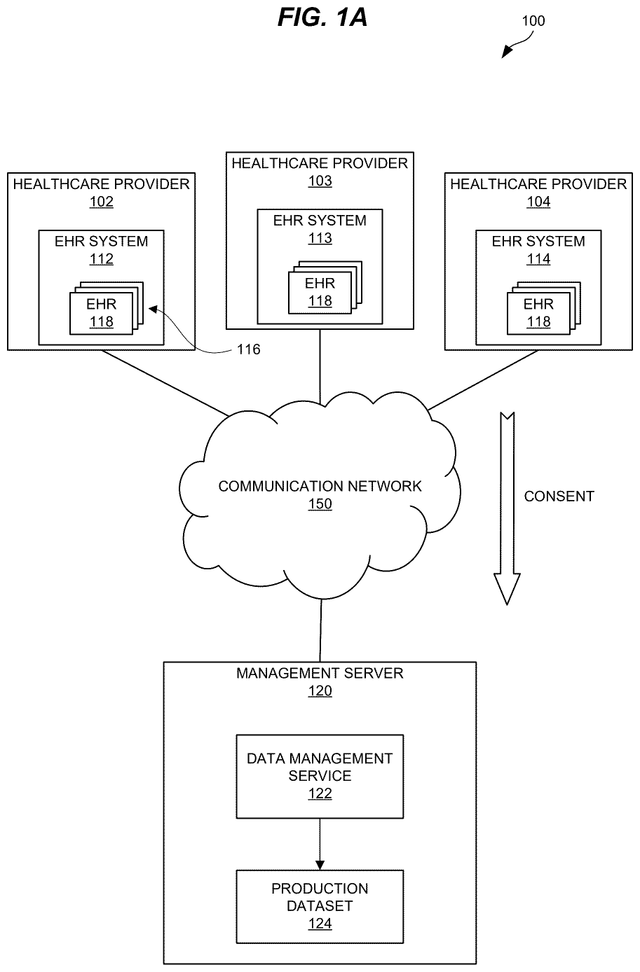

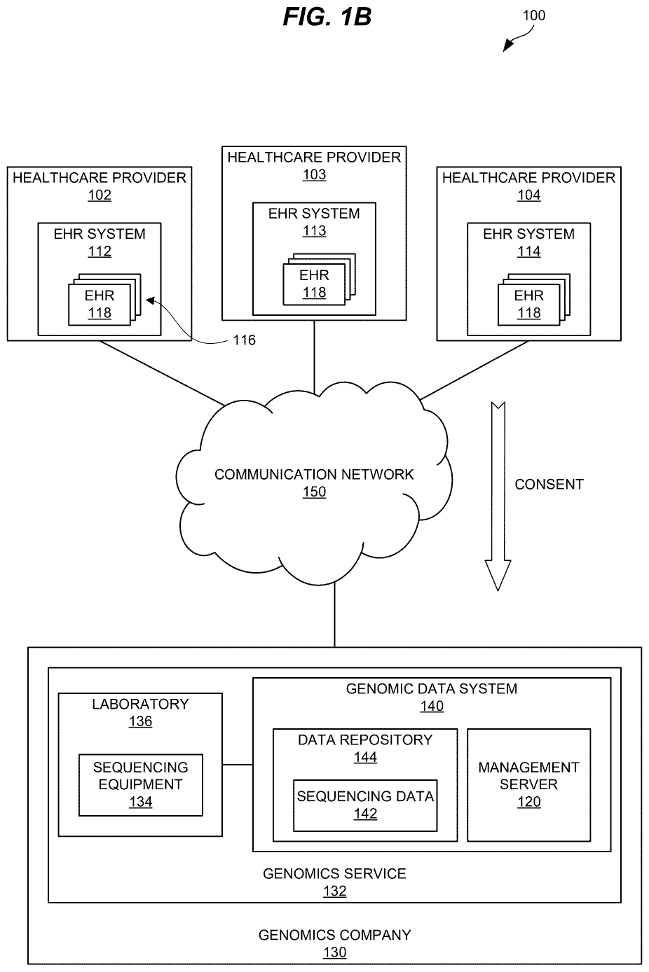

FIG. 1A is a block diagram of a health data management architecture 100 in an illustrative embodiment. At a high level, health data management architecture 100 comprises any combination of systems, components, and/or devices configured to compile and/or analyze health-related data (also referred to as healthcare data, health data, etc.). In an embodiment, health data management architecture 100 includes one or more EHR systems 112-114 (also referred to as EHR servers) of one or more healthcare providers 102-104 belonging to one or more healthcare provider networks (also referred to as a health partner, site, or care-site). A healthcare provider 102-104 is a licensed person or organization that provides healthcare services. One or more of the healthcare providers 102-104 may belong to a common healthcare provider network, or may belong to different healthcare provider networks. A healthcare provider 102-104 implements an EHR system 112-114 (e.g., an EPIC system) that maintains, tracks, and/or stores EHR data 116 for a plurality of patients. An Electronic Health Record (EHR) 118 is an electronic or digital version of a patient's medical history maintained by a healthcare provider or the like. EHR data 116 may include patient care information, including demographics (e.g., date of birth or age, gender, ethnicity, blood type, income, postal code, etc.), progress notes, problems, medications/prescriptions, vital signs, past medical history, immunizations, laboratory data or test results, radiology reports, medical codes (e.g., International Classification of Diseases (ICD) codes, Current Procedural Terminology (CPT) codes, etc.), and/or other information. An EHR system 112-114 may be implemented on a cloud-based or cloud-computing platform, and/or may be implemented on a hardware or server-based platform on-site for the healthcare provider 102-104.

Health data management architecture 100 further includes a management server 120, which is a data processing apparatus configured to gather, analyze, and/or process EHR data 116 and/or other health-related data for patients. Management server 120 may be configured to provide a data management service 122, which in general, has access to one or more databases of healthcare information, such as EHR data 116 maintained by one or more EHR systems 112-114, and/or other health-related data. The data management service 122 may be a fee-based service, such as a subscription-based service where a subscription is obtained to receive the data management service 122, a transaction-based service where a fee is charged per request or transaction, etc. As illustrated in FIG. 1A, management server 120 may have consent to access the EHR data 116 maintained by EHR systems 112-114, and/or other healthcare information. The data management service 122 may ingest health-related data (i.e., EHR data 116) from one or more health partners to generate a production dataset 124. The production dataset 124 is an aggregation or collection of health-related data that complies with certain rules or criteria, which may be used for further research, analysis, or some type of post-processing. In other words, management server 120 parses the incoming EHR data 116 for compliance with certain rules or criteria before addition to the production dataset 124 in order to assemble a robust production dataset 124.

Management server 120 is configured to communicate with external systems or devices via a communication network 150. Communication network 150 may comprise a Wide Area Network (WAN), such as the Internet, a telecommunications network, an enterprise network or private network, a Wireless Local Area Network (WLAN), etc., or any combination thereof. As will be described in more detail below, management server 120 is configured to receive or retrieve EHR data 116 stored in one or more EHR systems 112-114, via communication network 150. Management server 120 may be further configured to communicate with other external systems not shown, via communication network 150.

FIG. 1B is a block diagram of a health data management architecture 100 in another illustrative embodiment. In this embodiment, management server 120 may be implemented in or associated with a genomics service 132 offered by a genomics company 130. Genomics service 132 is in the field of bioinformatics, which is a scientific field related to the development or use of tools or applications to analyze and interpret biological data, such as DNA (deoxyribonucleic acid) sequences. At a high level, genomics service 132 comprises collection, storage, and/or analysis of genomic or genetic data. For the genomics service 132, genomics company 130 may perform or offer sample collection, DNA (deoxyribonucleic acid) or genomic sequencing, secure data storage of the sequencing data generated by sequencing processes, analysis of the sequencing data, etc. The genomics service 132 may be a fee-based service, such as a subscription-based service where a subscription is obtained to receive the genomics service 132.

For a sequencing process, genomics company 130 may implement or use sequencing equipment 134 (e.g., a sequencing instrument(s), a sequencing platform, a next-generation sequencing (NGS) platform, etc.) at a laboratory 136 or the like, which is configured to perform a sequencing process on biological samples. For example, DNA sequencing is a process of determining an exact sequence of nucleotides, or bases, in a DNA molecule. Sequencing equipment 134 may therefore include a DNA sequencer and/or other instruments configured to determine the order of the four bases: G (guanine), C (cytosine), A (adenine), and T (thymine). Genomic sequencing is a process of determining the entire genetic makeup of an organism.

Genomics company 130 may further implement a genomic data system 140 configured to store (i.e., secure data storage) sequencing data 142 (also referred to as genomic sequencing data or genetic sequencing data) in a data repository 144, analyze sequencing data 142, and/or otherwise manage sequencing data 142. For example, genomic data system 140 may process the sequencing data 142 (e.g., raw sequence data) to identify variants or alleles (i.e., variant calling). The sequencing data 142 as described herein may include raw DNA or genomic sequences (e.g., order of the bases), and any associated data extracted from the raw sequences, such as aligned sequence data, variant information or variant call data, etc. Genomic data system 140 may be implemented at a laboratory 136 of the genomics company 130, such as on servers or other on-premises resources at the laboratory 136. Alternatively, genomic data system 140 may be implemented on one or more external platforms, such as a cloud infrastructure of a cloud computing platform. Cloud computing is the delivery of computing resources, including storage, processing power, databases, networking, analytics, artificial intelligence, and software applications, over an internet connection. Some examples of a cloud computing platform may comprise Amazon Web Services (AWS), Google Cloud, Microsoft Azure, etc. Further, although genomics company 130 is illustrated as implementing sequencing equipment 134 and genomic data system 140, it is understood that the sequencing equipment 134 and genomic data system 140 may be distributed among different companies, entities, platforms, etc.

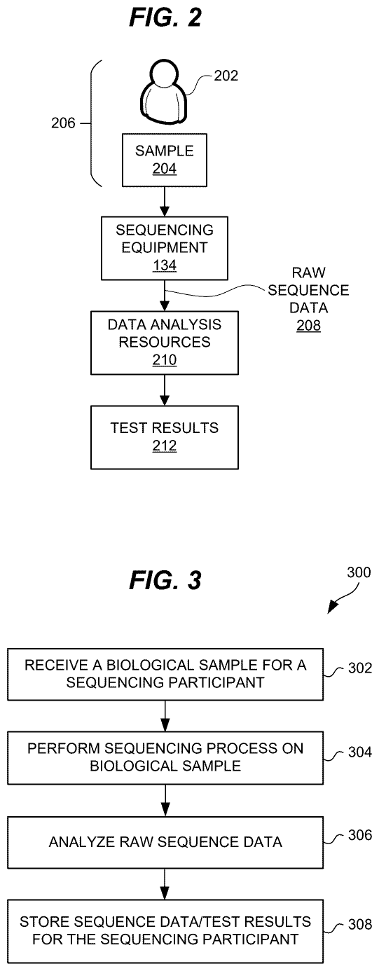

FIG. 2 is a block diagram illustrating genetic testing in an illustrative embodiment. FIG. 3 is a flow chart illustrating a method 300 of genetic testing in an illustrative embodiment. The steps of the flow charts described herein are not all inclusive and may include other steps not shown, and the steps may be performed in an alternative order. A biological sample 204 (e.g., blood, saliva, etc.) of an individual 202 is received at laboratory 136 for sequencing (step 302). An individual 202 that volunteers or consents to genomic sequencing of a biological sample 204 is referred to as a sequencing participant 206. The sequencing equipment 134 at laboratory 136 performs a sequencing process on the biological sample 204 to generate raw sequence data 208 associated with the sequencing participant 206 (step 304). Data analysis resources 210 may then analyze or otherwise process the raw sequence data 208, such as alignment, variant calling, and/or any other analysis (step 306). The analysis process generates test results 212 (also referred to as diagnostic results, analysis results, genomic analysis results, analysis output, etc.). The raw sequence data 208 and any data or information generated by the analysis of the raw sequence data 208, such as the aligned sequence data, variant information or variant call data, etc., may be collectively referred to as sequencing data 142 for, or associated with, a sequencing participant 206. The sequencing data 142 may comprise data for a whole genome, a subset of the genes that make up a genome, etc. The sequencing data 142 and/or test results 212 are stored in secure data storage (step 308), such as in a data repository.

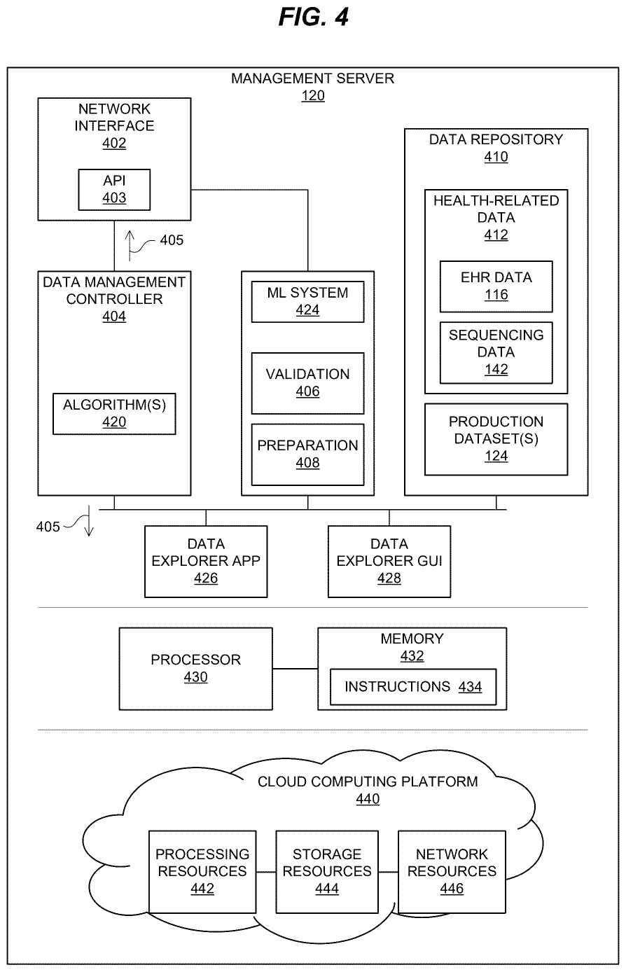

FIG. 4 is a block diagram of management server 120 in an illustrative embodiment. Management server 120 may include the following subsystems: a network interface component 402 (also referred to as a network interface), a data management controller 404, a validation unit 406, a preparation unit 408, and a data repository 410 that operate on one or more platforms. Network interface component 402 may comprise circuitry, logic, hardware, means, etc., configured to exchange messages, documents, and/or electronic data communications with external devices or systems. Network interface component 402 may operate using a variety of protocols and/or Application Programming Interfaces 403 (APIs). Data management controller 404 may comprise circuitry, logic, hardware, means, etc., configured to control, direct, or supervise the ingestion and/or processing of EHR data 116, sequencing data 142, and/or other health-related data 412 or patient data, analyzing of the health-related data 412 to extract insights, and/or performing of other functions within management server 120. Data management controller 404 may execute one or more algorithms 420 (also referred to as scripts or control files (e.g., Python files containing Directed Acyclic Graphs (DAGs) or files of another programming language) to perform its functions, and output control signals 405 to other system(s). Validation unit 406 is a processing unit, module, system, circuitry, logic, hardware, means, etc., configured to validate EHR data 116 and/or other health-related data 412 or patient data received from health partners or the like, and/or perform other functions. Preparation unit 408 is a processing unit, module, system, circuitry, logic, hardware, means, etc., configured to prepare (e.g., transform, merge, and/or annotate) validated or compliant EHR data 116 and/or other health-related data 412 or patient data for storage or inclusion in production dataset 124. Management server 120 may implement one or more machine learning (ML) systems 424 to perform one or more actions or tasks as described herein, such as for validation unit 406, preparation unit 408, etc. Management server 120 or data management controller 404 may execute a data explorer application 426 to perform the functions or operations, such as analyzing or exploring the production dataset 124. Management server 120 may also provide a data explorer Graphical User Interface (GUI) 428, which is a digital interface configured to interact with a user. Data repository 410 comprises secure data storage configured to store health-related data 412 (e.g., EHR data 116, sequencing data 142, etc.), one or more production datasets 124, and/or other data.

One or more of the subsystems of management server 120 may be implemented on a hardware platform comprised of analog and/or digital circuitry. For example, network interface component 402, data management controller 404, validation unit 406, and/or preparation unit 408 may be implemented on one or more processors 430 that execute instructions 434 (i.e., computer readable code) for software that are loaded into memory 432. A processor 430 comprises an integrated hardware circuit configured to execute instructions 434 to provide the functions of management server 120. Processor 430 may comprise a set of one or more processors or may comprise a multi-processor core, depending on the particular implementation. Memory 432 is a non-transitory computer readable storage medium for data, instructions, applications, etc., and is accessible by processor 430. Memory 432 is a hardware storage device capable of storing information on a temporary basis and/or a permanent basis. Memory 432 may comprise a random-access memory, or any other volatile or non-volatile storage device.

One or more of the subsystems of management server 120 may be implemented on cloud computing platform 440 (e.g., AWS) or another type of processing platform. Cloud resources may be provisioned on cloud computing platform 440, such as processing resources 442 (e.g., physical or hardware processors, a server, a virtual server or virtual machine (VM), a virtual central processing unit (vCPU), etc.), storage resources 444 (e.g., physical or hardware storage, virtual storage, etc.), and/or networking resources 446, although other resources are considered herein. Management server 120 may be built upon the provisioned resources with instructions, programming, code, etc. For example, network interface component 402 may be provisioned on networking resources 446, data management controller 404, validation unit 406, and/or preparation unit 408 may be provisioned on processing resources 442, and data repository 410 may be provisioned on storage resources 444.

Management server 120 may include various other components not specifically illustrated in FIG. 4.

In embodiments described herein, management server 120 is configured to manage EHR data 116 from one or more health partners to generate one or more production datasets 124 that may be used for further research, analysis, etc. In other words, the production dataset 124 comprises a collection of EHR data 116 that is verified in terms of structure, content, accuracy, etc., and is considered a trustworthy dataset that may be used for further research, analysis, etc. One technical benefit is the production dataset 124 comprises a rich data set for a large population from which insights may be determined to better understand relationships between various health conditions for patients, demographics, genomics/genetics, etc.

As a general overview, management server 120 receives a batch of EHR data 116 from a health partner, and performs an initial validation of the EHR data 116 to determine whether the EHR data 116 complies or conforms with a target structure or data model (i.e., contains the desired tables, fields, etc.). Management server 120 may then perform subsequent validation of the EHR data 116 to verify the accuracy or quality of the content contained in the EHR data 116. When the EHR data 116 passes validation, the EHR data 116 may be stored or added to the production dataset 124. Management server 120 also generates a report(s) of the incoming EHR data 116 (e.g., indicating non-compliant data, issues, errors, inaccuracies, metrics, etc.) that is reported back to the health partner. One technical benefit is the veracity of the production dataset 124 is improved by validating the EHR data 116 that is added. Another technical benefit is the management server 120 is able to report issues found in the EHR data 116 submitted by a health partner, which may be used to improve future batches or re-submissions of EHR data 116.

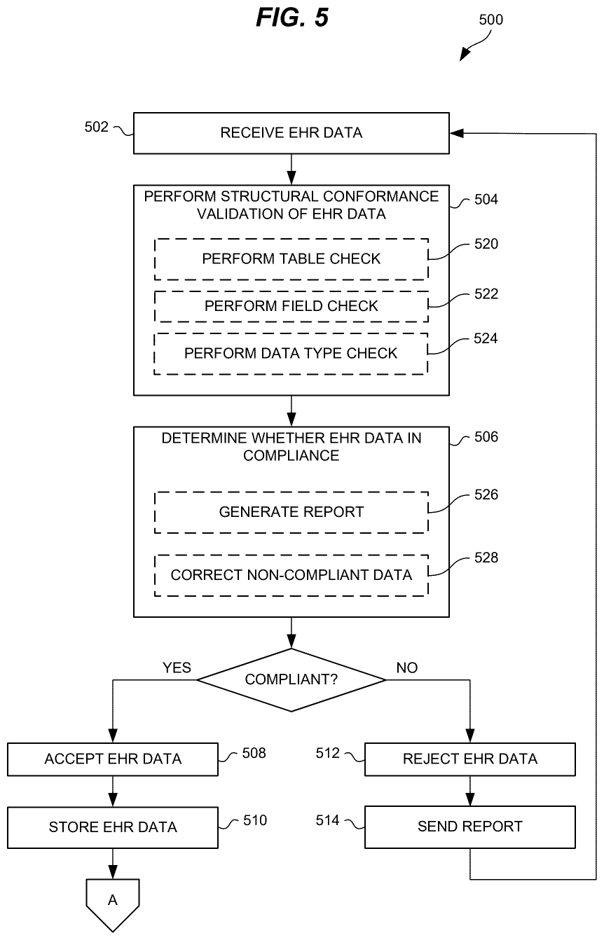

FIG. 5 is a flow chart illustrating a method 500 of managing EHR data 116 in an illustrative embodiment. The steps of method 500 will be described with reference to management server 120 in FIG. 4, but those skilled in the art will appreciate that method 500 may be performed in other systems or devices.

Management server 120 ingests, inputs, obtains, or receives EHR data 116 (step 502) from a health partner during an ingestion phase (also referred to as an ingestion service). For example, data management controller 404 may output a control signal 405 to network interface component 402 to receive the EHR data 116 from a health partner. Management server 120 may receive the EHR data 116 through an API, over a protocol such as sFTP (secure File Transfer Protocol), by accessing a Uniform Resource Locator (URL) through an HTTPS (Hypertext Transfer Protocol Secure) connection or the like, etc.

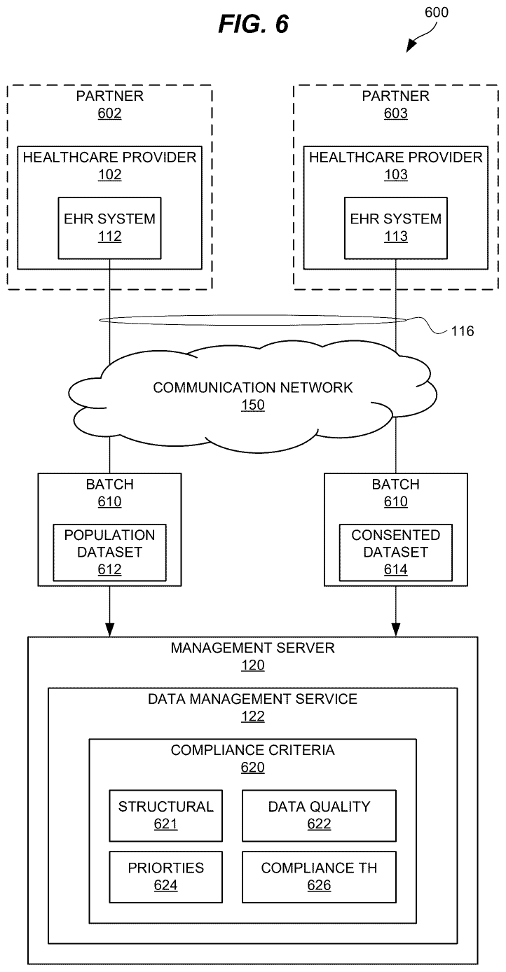

FIG. 6 illustrates an ingestion phase 600 of EHR data 116 into the data management service 122 in an illustrative embodiment. In this example, management server 120 may ingest EHR data 116 from one or more health partners 602-603 (e.g., healthcare provider networks), such as over a communication network 150. Management server 120 may receive EHR data 116 in a batch 610 from a health partner 602-603 for a number of patients, which may be referred to as batched EHR data or batched EHRs. In operation, a batch 610 is a voluminous amount of EHR data 116 ingested at a time, such as for a number of patients or number of records that exceeds a minimum threshold (e.g., ten thousand patients/records, one hundred thousand patients/records, one million patients/records, etc.). In an embodiment, a batch 610 of EHR data 116 may be pushed from an EHR system(s) 112-113 or health partner 602-603 periodically (e.g., weekly, bi-weekly, monthly, quarterly, etc.), a batch 610 of EHR data 116 may be pulled from an EHR system(s) 112-113 or health partner 602-603 in response to a request, etc.

In an embodiment, management server 120 may receive a batch 610 of EHR data 116 referred to as a population dataset 612. Population dataset 612 is a collection of EHR data from a health partner regarding a population of patients served by the health partner. For example, a population dataset 612 may comprise EHR data for each or all of the patients served by the health partner 602, for which an EHR 118 is recorded in an EHR system 112. The EHR data 116 may be anonymized so that patients are not individually identifiable. In an embodiment, management server 120 may receive a batch 610 of EHR data 116 referred to as a consented dataset 614. Consented dataset 614 is a collection of EHR data from a health partner regarding a group of sequencing participants 206. For example, a subset of patients served by a health partner 603 may have volunteered or consented to genomic sequencing. Thus, sequencing data 142 and/or any associated test results may be generated (or will be generated) for the sequencing participants 206, which is associated with the EHR data 116 (i.e., information included in the EHR data 116 or linked to the EHR data 116). The EHR data 116 associated with sequencing participants 206 may be handled separately as a consented dataset 614.

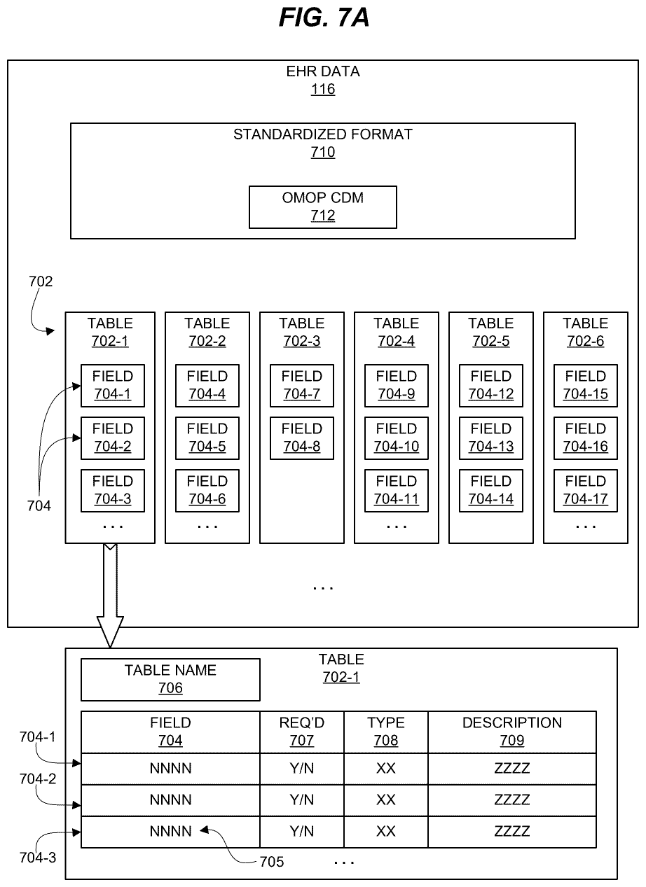

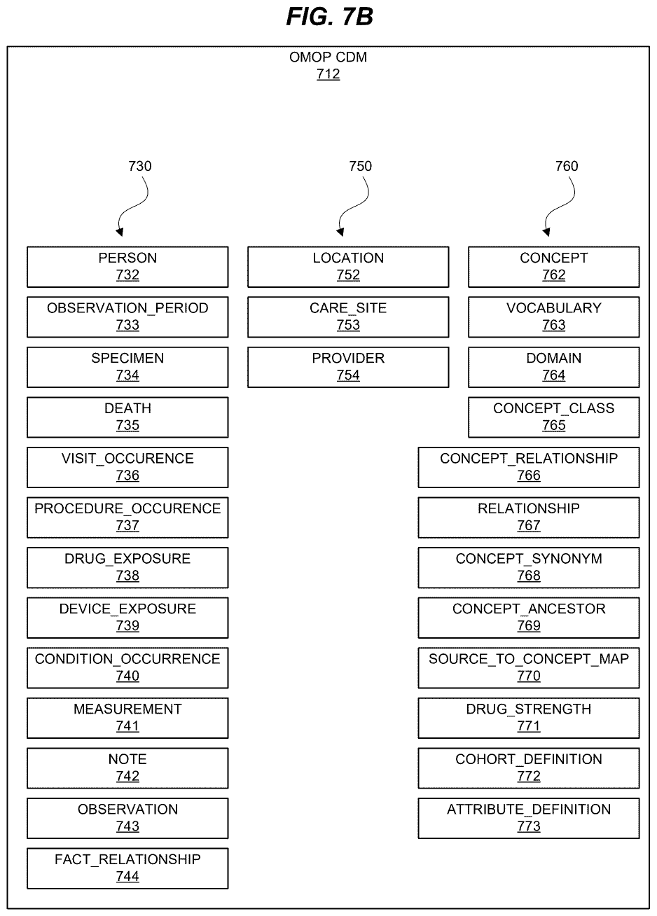

FIG. 7A illustrates EHR data 116 in an illustrative embodiment. The EHR data 116 received from a health partner 602-603 may be in a standardized format 710, such as OMOP CDM format 712. OMOP CDM is a standard designed to standardize the structure and content of observational data, such as EHR data 116. In general, EHR data 116 in a standardized format 710 may include a number of tables 702 and a number of fields 704. In database parlance, tables 702 and fields 704 as described herein may be referred to as columns and rows, respectively. As in FIG. 7A, EHR data 116 may include a plurality of tables 702 (e.g., table 702-1, 702-2, 702-3, etc.), with one or more fields 704 (e.g., 704-1, 704-2, 704-3, etc.) defined within each table 702 (it is noted that one or more tables may be nested within another table). Each table 702, for example, may include a table name 706, and one or more associated fields 704 (and/or nested tables). Each field 704, for example, may be identifiable by a field name or identifier, and may include a value 705 (which may be referred to as a table value or field value), a required indication 707 indicating whether or not the field 704 is required, a data type 708 (e.g., integer, floating point, character, string, Boolean, enumerated type, array, date, etc.) of the value 705, a field description 709, etc.

In other embodiments, the EHR data 116 received from a health partner 602-603 may be in a non-standardized or customized format, and management server 120 may convert the EHR data 116 to a standardized format 710 in the ingestion phase 600.

FIG. 7B illustrates OMOP CDM format 712 in an illustrative embodiment. OMOP CDM format 712 includes a plurality of tables 702 and a plurality of fields 704. As an example, OMOP CDM format 712 may include a plurality of clinical data tables 730, such as a person table 732, an observation_period table 733, a specimen table 734, a death table 735, a visit_occurrence table 736, a procedure_occurrence table 737, a drug_exposure table 738, a device_exposure table 739, a condition_occurrence table 740, a measurement table 741, a note table 742, an observation table 743, a fact_relationship table 744, etc. OMOP CDM format 712 may include a plurality of health system data tables 750, such as location table 752, a care_site table 753, a provider table 754, etc. OMOP CDM format 712 may include a plurality of vocabularies tables 760, such as a concept table 762, a vocabulary table 763, a domain table 764, a concept_class table 765, a concept_relationship table 766, a relationship table 767, a concept_synonym table 768, a concept_ancestor table 769, a source_to_concept_map table 770, a drug strength table 771, a cohort definition table 772, an attribute_definition table 773, etc. It is noted that FIG. 7B is a representative example of OMOP CDM format 712, and any changes or updates to the OMOP CDM format 712 are considered herein.

Although the EHR data 116 may be in a standardized format, such as OMOP CDM format 712, the EHR data 116 from a health partner 602-603 may exclude one or more tables 702 and/or fields 704, one or more tables 702 and/or fields 704 may be used for different purposes by health partners 602-603, one or more tables 702 and/or fields 704 may be provisioned with different data or data types, etc. For example, health partners 602-603 may use different table names 706 and/or field IDs, the content of tables 702 and/or the fields 704 may be different, etc. Before storing EHR data 116 as part of the production dataset 124, it may be beneficial to ensure that the EHR data 116 complies with a set of requirements for the format and/or content of the data. Thus, management server 120 may be provisioned with local policies or rules (e.g., stored in memory 432) referred to as compliance criteria 620 (see FIG. 6), which may be used to validate the EHR data 116. The compliance criteria 620 may comprise structural compliance criteria 621 used to validate the structure of the EHR data 116, and data quality compliance criteria 622 used to validate the quality of the EHR data 116.

In FIG. 5, management server 120 performs structural conformance validation (also referred to as a structural conformance validation service or OMOP CDM conformance validation) of the EHR data 116 based on the structural compliance criteria 621 (step 504). For example, data management controller 404 may output a control signal 405 to validation unit 406 to perform structural conformance validation on the EHR data 116 of the batch 610, as validation may be performed on a batch-by-batch basis. Validation unit 406 may automatically perform structural conformance validation in response to receipt of a batch 610 of EHR data 116 and/or in response to a control signal 405 from data management controller 404. In an embodiment, validation unit 406 may compare the incoming EHR data 116 of the batch 610 to a target EHR structure or data model defined in the structural compliance criteria 621. For example, a data model may be provided to health partners 602-603 indicating a desired or model data structure for EHR data 116. When ingesting a new batch 610, validation unit 406 may parse or otherwise perform electronic data processing on the EHR data 116 to compare the EHR data 116 with the structural compliance criteria 621 to determine whether the EHR data 116 of the batch 610 is compliant with the data model. The structural compliance criteria 621 sets forth policies or rules of the data model for EHR data 116 received from a health partner 602-603 and approved for inclusion in the production dataset 124. One technical benefit is the structural conformance validation may identify any inaccurate, incomplete, and/or corrupted data to ensure the veracity of the production dataset 124.

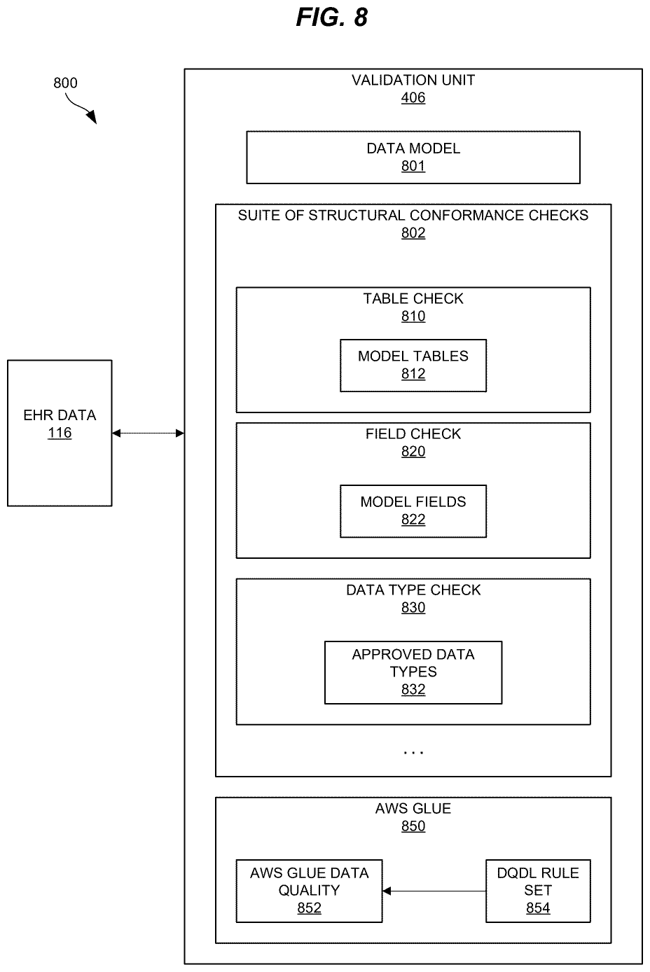

FIG. 8 is a block diagram illustrating structural conformance validation 800 in an illustrative embodiment. As part of the structural compliance criteria 621, a data model 801 may be defined that comprises a model data structure for EHR data 116 that represents an example for a health partner to follow or imitate. In general, validation of EHR data 116 may use a suite of structural conformance checks 802 based on the compliance criteria 620. For one of the structural conformance checks 802, management server 120 may perform a table check 810 (or column check) to determine whether table data (i.e., the tables 702) of the received EHR data 116 is in conformance with the structural compliance criteria 621 or data model 801 (see optional step 520 in FIG. 5). For example, the data model 801 may indicate a set of model tables 812 (e.g., fourteen to sixteen) that are required or expected in EHR data 116. The table check 810 may determine whether the tables 702 of EHR data 116 comply with the set of model tables 812 required or expected in EHR data 116. For example, validation unit 406 may determine whether the number of tables 702 complies with the data model 801, whether one or more tables 702 are missing or mandatory tables 702 are present, whether the tables 702 are labeled correctly (i.e., table name 706), whether the tables 702 are arranged in the correct order, etc. The table check 810 may determine the validity of the table data (i.e., of values 705 in a table 702). For example, validation unit 406 may determine a percentage of non-null values in a table 702 to ensure values 705 of a given table 702 do not have an unacceptable percentage of null values, determine whether values 705 of a table 702 conform to a given expression, determine whether the character length of string values 705 of a table 702 conforms with a character limit, etc.

For another one of the structural conformance checks 802, management server 120 may perform a field check 820 (or row check) to determine whether field data (i.e., fields 704) of the received EHR data 116 is in conformance with the structural compliance criteria 621 or data model 801 (see optional step 522 in FIG. 5). For example, the data model 801 may indicate a set of model fields 822 that are required or expected in EHR data 116. The field check 820 determines whether the fields 704 of EHR data 116 comply with the set of model fields 822 required or expected in EHR data 116. For example, validation unit 406 may determine whether the number of fields 704 complies with the data model 801, whether one or more fields 704 are missing or mandatory fields 704 are present, whether the fields 704 are labeled correctly (i.e., field IDs), whether the fields 704 are arranged in the correct order, etc.

For another one of the structural conformance checks 802, management server 120 may perform a data type check 830 (see optional step 524 in FIG. 5). In the data type check 830, validation unit 406 validates or verifies data types for the values 705 (or a subset thereof) provisioned in the EHR data 116 based on the structural compliance criteria 621. In other words, validation unit 406 determines whether a data type 708 of values 705 provisioned in one or more tables 702 or fields 704 of EHR data 116 complies with the structural compliance criteria 621. The structural compliance criteria 621 may include rules of approved data types 832 (e.g., integer, floating point, character, string, Boolean, enumerated type, array, date, etc.) for values 705 of the EHR data 116, and validation unit 406 may compare the data types 708 (see FIG. 7A) for the values 705 of the EHR data 116 with the approved data types 832 for validation. For example, validation unit 406 may determine whether a value 705 populated in a field 704 for a dosage of medication is not specified as a “date” data type, when the data type 708 for the field 704 is actually defined as a “floating point” data type.

The suite of structural conformance checks 802 may include additional or alternative checks as desired, such as a check that a file provided by a health partner is not empty (e.g., file size is greater than 0 bytes), that the file is in a supported file format (e.g., file format is not in .csv, .tsv, metadata.json, or .parquet format), and/or other checks. One technical benefit is management server 120 verifies the structure and completeness of the EHR data 116 during the ingestion phase 600.

In an embodiment, validation unit 406 may be implemented in the AWS Glue service 850. A feature of the AWS Glue service 850 is AWS Glue Data Quality 852, which is a serverless service that allows a user to measure and/or monitor the quality of data. The AWS Glue Data Quality 852 evaluates objects (e.g., EHR data 116) stored in the AWS Glue Data Catalog, and performs or enforces data quality checks on the objects. For the AWS Glue Data Quality 852, a Data Quality Definition Language (DQDL) rule set 854 is defined. DQDL is a domain specific language for defining rules for AWS Glue Data Quality 852. The DQDL rule set 854 is an example of the compliance criteria 620, and sets out the rules used to evaluate EHR data 116 for structural conformance.

In FIG. 5, management server 120 determines whether the EHR data 116 of the batch 610 is in compliance with the structural compliance criteria 621 according to or based on the structural conformance validation 800 (step 506). For example, data management controller 404 may output a control signal 405 to validation unit 406 to determine whether the EHR data 116 of a batch 610 is in compliance with the structural compliance criteria 621. Management server 120 may detect or identify any errors or issues (i.e., non-compliant data) in the EHR data 116, and determine whether the errors or issues exceed one or more compliance thresholds. As indicated in FIG. 6, the compliance criteria 620 may define levels or priorities 624 of errors detected in the EHR data 116, such as “critical”, “moderate”, and “low” priority. For any errors or issues detected in the EHR data 116, management server 120 may determine whether the EHR data 116 passes the structural conformance validation 800 based on the compliance thresholds 626. For example, certain errors may not have a significant impact on the utilization of the EHR data 116 (e.g., low priority errors), while other errors (e.g., critical or moderate errors) may have an impact on the utilization of the EHR data 116. The compliance thresholds 626 may therefore define or specify which errors are acceptable and which errors are not.

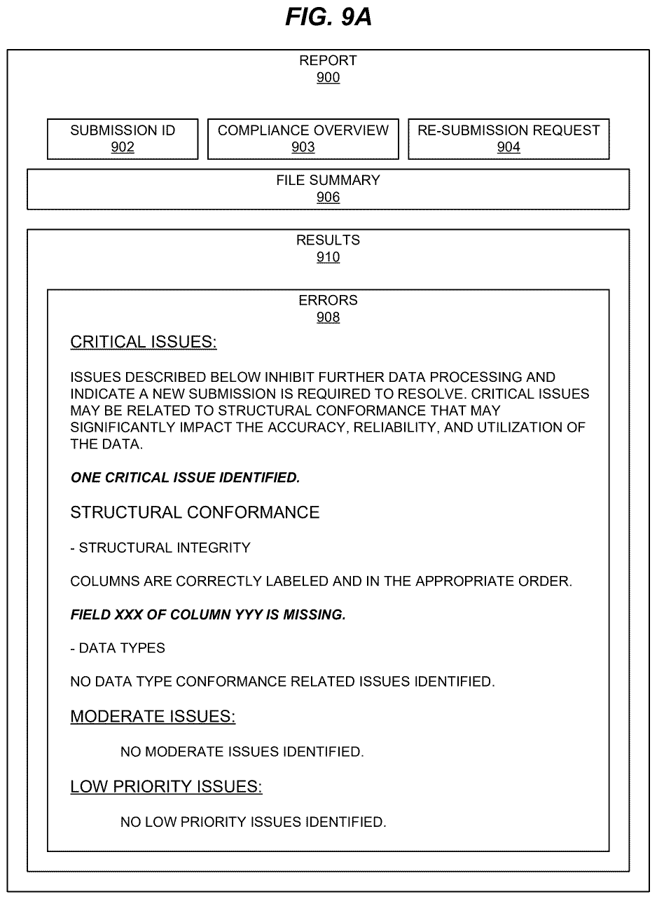

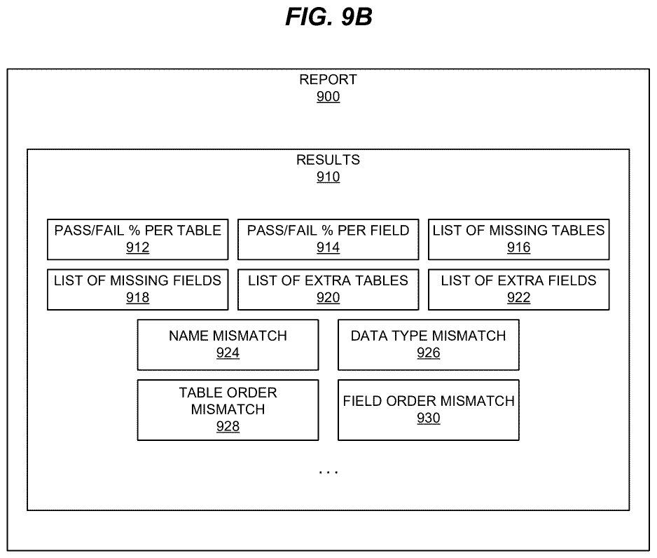

In an embodiment, management server 120 may generate a report based on the structural conformance validation 800 (optional step 526). For example, data management controller 404 may output a control signal 405 to validation unit 406 to generate a report indicating results of the structural conformance validation 800. FIGS. 9A-9B illustrate a report 900 in an illustrative embodiment. The report 900 comprises information, statistics, etc., regarding differences, errors, or issues detected in the EHR data 116 based on the structural conformance validation 800. In FIG. 9A, the report 900 may include a submission identifier (ID) 902 indicating the batch 610 of EHR data 116 submitted by a health partner 602-603. The report 900 may include an overview 903 highlighting any errors or issues encountered while processing the batch 610 of EHR data 116 indicated by the submission ID 902. The report 900 may include a re-submission request 904 for a health partner 602-603 to re-submit a corrected or modified dataset (i.e., a corrected version of the EHR data 116), such as when critical errors have been identified. The report 900 may include a file summary 906 of the file(s) that were received or examined. The report 900 includes results 910 of the structural conformance validation 800. The results 910 may include or indicate one or more errors 908 detected in the EHR data 116, may indicate a priority 624 of the error(s) 908 (e.g., “critical”, “moderate”, and “low” priority), and/or other information. An example of the results 910 is provided in FIG. 9A merely as an example, and the content of the results 910 may vary as desired. In FIG. 9B, the results 910 may include a pass or fail percentage 912 per table, a pass or fail percentage 914 per field, a list 916 of missing tables, a list 918 of missing fields, a list 920 of extra tables, a list 922 of extra fields, table and/or field name mismatch information 924, data type mismatch information 926, table order mismatch information 928, field order mismatch information 930, etc. One technical benefit is the report 900 provides feedback to a health partner 602-603 regarding one or more errors 908 found in the EHR data 116. The health partner 602-603 may therefore fix any errors in a re-submission and/or future submissions.

In an embodiment, management server 120 may correct one or more errors 908 (e.g., non-compliant data) detected in the EHR data 116 (optional step 528 in FIG. 5), such as based on the priority 624 of the errors 908. According to the compliance criteria 620, certain errors 908 may be automatically corrected by the management server 120, such as low priority errors. For example, the data type 708 for one or more values 705 may be modified to the correct data type based on the compliance criteria 620. Further, management server 120 may seek feedback from a human, a domain expert, or the like to correct certain errors 908.

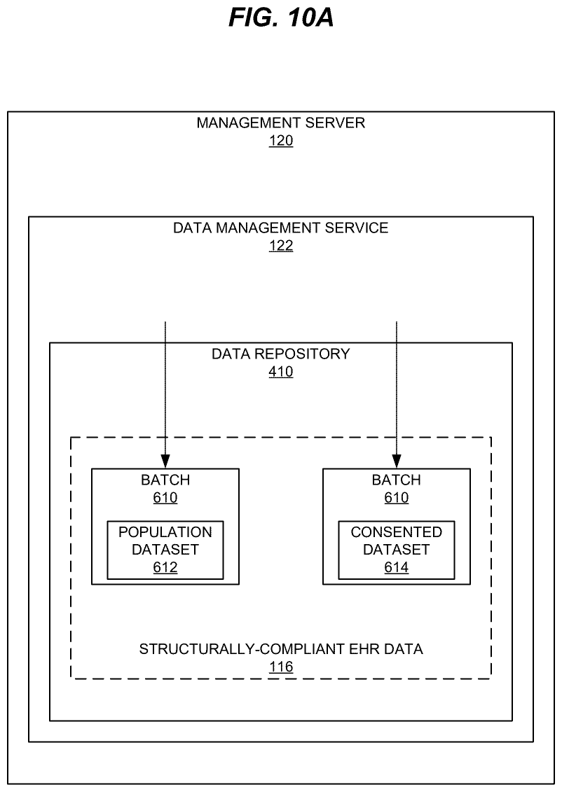

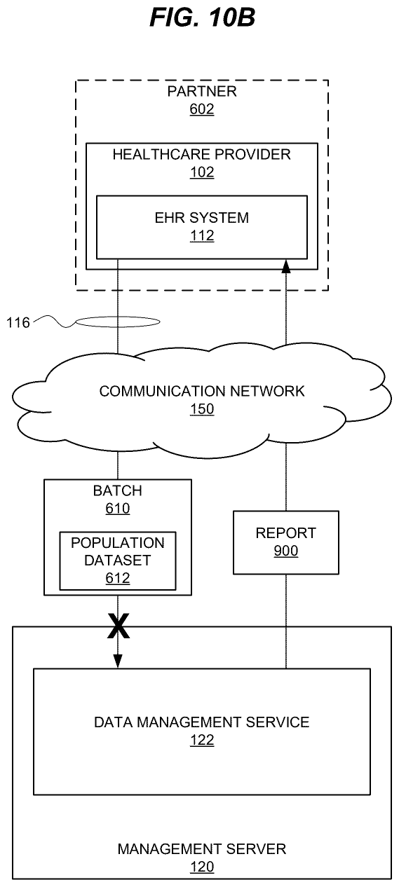

Based on the determination in step 506, management server 120 accepts the EHR data 116 of the batch 610 from a health partner 602-603 or rejects the EHR data 116. More particularly, when EHR data 116 of the batch 610 is compliant based on the structural conformance validation 800 (i.e., in compliance with the structural compliance criteria 621), management server 120 accepts the EHR data 116 as structurally-compliant EHR data 116 (step 508), and stores the structurally-compliant EHR data 116 in the data repository 410 (step 510). For example, data management controller 404 may output a control signal 405 to validation unit 406 to store the structurally-compliant EHR data 116 in data repository 410. FIG. 10A illustrates accepting of the EHR data 116 during the ingestion phase 600 in an illustrative embodiment. In this example, the EHR data 116 of the batch 610 is compliant based on the structural conformance validation 800. Thus, management server 120 stores the EHR data 116 of the batch 610 in the data repository 410 as structurally-compliant EHR data. One technical benefit is management server 120 verifies that the EHR data 116 received from a health partner 602-603 conforms to a desired data model 801 when ingesting the data.

In FIG. 5, when EHR data 116 of the batch 610 is non-compliant based on the structural conformance validation 800 (i.e., not in compliance with the structural compliance criteria 621), management server 120 rejects the EHR data 116 as non-compliant EHR data 116 (step 512), and sends a report 900 to the health partner 602-603 (step 514). For example, data management controller 404 may output a control signal 405 to network interface component 402 to send the report 900 to the health partner 602-603. In an embodiment, management server 120 may send a re-submission request 904 to the health partner 602-603 in the report 900, separate from the report 900, etc., to submit a modified batch 610 of EHR data 116. In another embodiment, management server 120 may wait for the next submission from the health partner 602-603 that is modified based on the report 900. FIG. 10B illustrates rejecting of the EHR data 116 during the ingestion phase 600 in an illustrative embodiment. In this example, the EHR data 116 of the batch 610 is not compliant based on the structural conformance validation 800. Thus, management server 120 rejects the EHR data 116 (i.e., does not store the EHR data 116 of the batch 610 in the data repository 410), and sends the report 900 to the health partner 602-603 with a re-submission request 904. One technical benefit is management server 120 reports non-compliant EHR data 116 to a health partner 602-603 to allow the health partner 602-603 to submit a corrected dataset, which improves the quality of the production dataset 124 that is compiled from the EHR data 116.

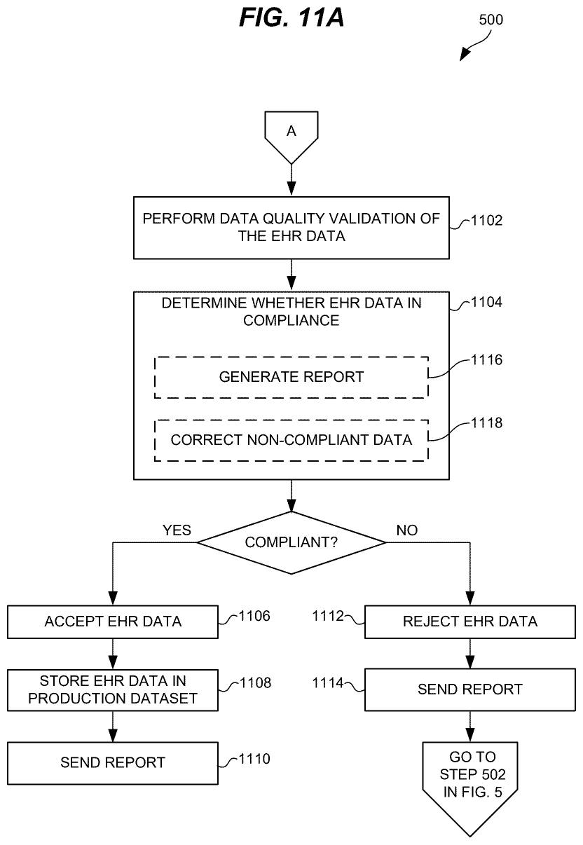

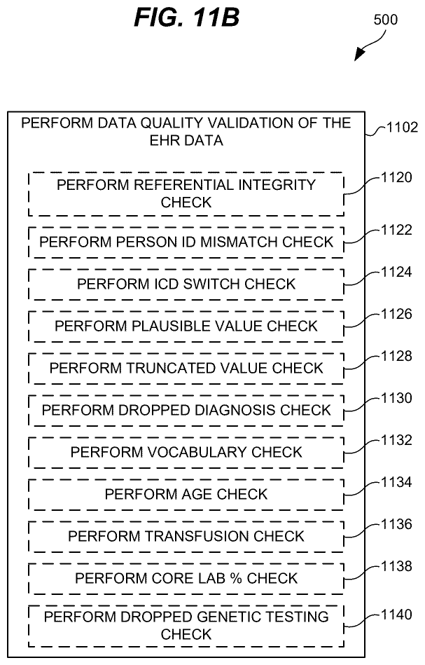

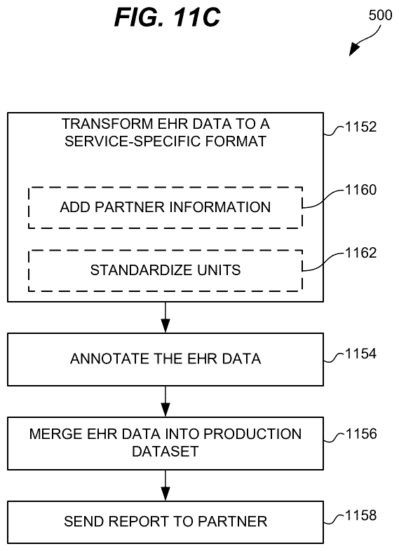

After the ingestion phase 600 of the batch 610 of EHR data 116, management server 120 may perform further validation of the content of the structurally-compliant EHR data 116, which is referred to as data quality validation. FIGS. 11A-11C are flow charts illustrating additional steps of the method 500 for managing EHR data 116 in illustrative embodiments. In FIG. 11A, management server 120 performs data quality validation (also referred to as a data quality validation process or data quality validation service) of the structurally-compliant EHR data 116 that passed structural conformance validation 800 (step 1102). For example, data management controller 404 may output a control signal 405 to validation unit 406 to load the structurally-compliant EHR data 116 from data repository 410, and perform data quality validation on the structurally-compliant EHR data 116. Validation unit 406 may parse or otherwise perform electronic data processing on the structurally-compliant EHR data 116 to validate the content of values 705 contained in the structurally-compliant EHR data 116. Whereas structural conformance validation 800 compares EHR data 116 with a data model 801, data quality validation examines the values 705 within the structurally-compliant EHR data 116 to identify any inconsistencies, issues, errors, etc.

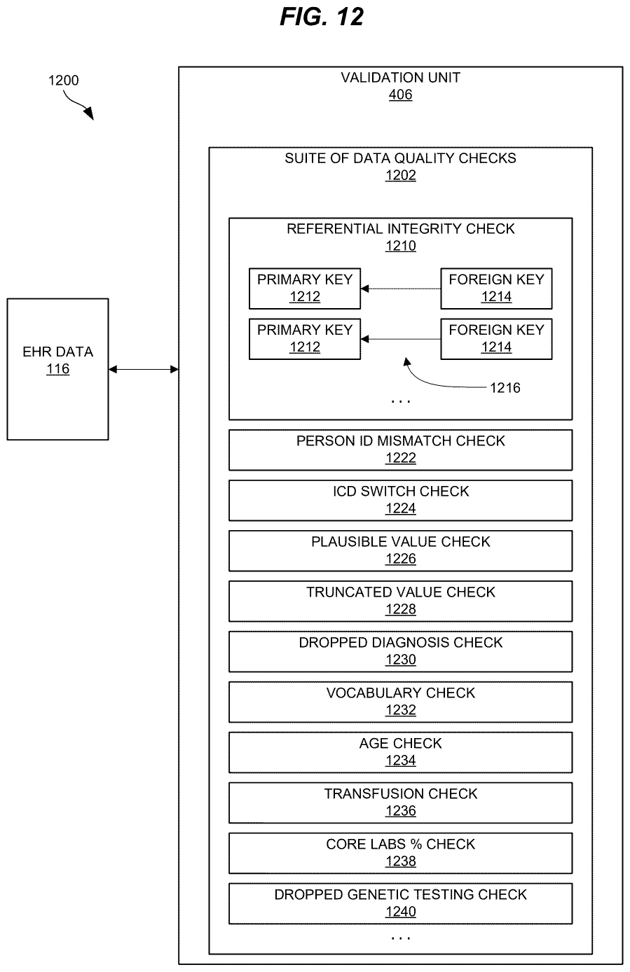

FIG. 12 is a block diagram illustrating data quality validation 1200 in an illustrative embodiment. In general, validation of structurally-compliant EHR data 116 may use a suite of data quality checks 1202. For one of the data quality checks 1202, management server 120 may perform a referential integrity check 1210 (see optional step 1120 in FIG. 11B). In a referential integrity check 1210, validation unit 406 evaluates whether relationships between fields 704 of the EHR data 116 are valid. The referential integrity check 1210 may compare foreign key values with primary key values to identify any violations. A primary key 1212 is a unique identifier for each field 704 in a table 702, and a foreign key 1214 is a field 704 in one table 702 that refers to the primary key 1212 in another table 702. The referential integrity check 1210 determines, validates, or ensures that the relationships 1216 or mappings between fields 704 (i.e., between foreign keys 1214 and primary keys 1212) remain valid and consistent. The referential integrity check 1210 determines or ensures that a foreign key 1214 in one table 702 points to an existing, valid primary key 1212 in another table 702, which prevents orphaned fields or broken links within the data.

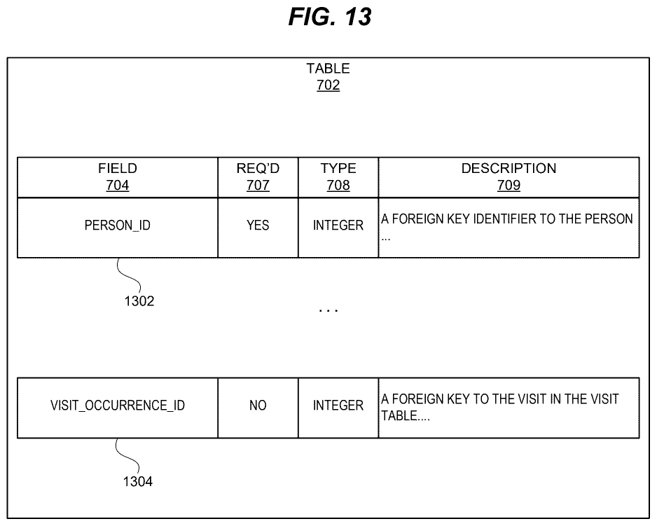

For another one of the data quality checks 1202 in FIG. 12, management server 120 may perform person ID mismatch check 1222 (see optional step 1122 in FIG. 11B). In EHR data 116, multiple tables 702 may include or reference a person ID and a visit ID. In a person ID mismatch check 1222, validation unit 406 determines whether each table 702 provisioned with a person ID and a visit ID maps the visit ID to the same person ID as the other tables 702. FIG. 13 illustrates a table 702 in an illustrative embodiment. The table 702 in FIG. 13 may comprise a visit_occurrence table 736, a procedure_occurrence table 737, a drug_exposure table 738, a device_exposure table 739, a condition_occurrence table 740, a measurement table 741, a note table 742, an observation table 743, etc. The table 702 includes a field 704 that indicates a person ID 1302 (e.g., person_id) and a visit ID 1304 (e.g., visit_occurrence_id). Validation unit 406 may query the tables 702 to determine whether a mapping between the person ID 1302 and the visit ID 1304 is consistent across the tables 702 (e.g., the same person ID 1302 is mapped to the same visit ID 1304).

For another one of the data quality checks 1202 in FIG. 12, management server 120 may perform an ICD switch check 1224 (see optional step 1124 in FIG. 11B). The International Classification of Diseases (ICD) is used to standardize codes for medical conditions and procedures. The 9^{th} revision of these standardized codes (ICD-9) was replaced with the 10^{th} revision (ICD-10) in the year 2015, making ICD-9 out of date. The ICD switch check 1224 determines whether codes used in the EHR data 116 are ICD-10 codes. For example, codes (i.e., medical code or diagnostic codes) are recorded in a condition occurrence table (e.g., condition_occurrence table 740). Thus, validation unit 406 may query the condition occurrence table to determine whether codes recorded in the condition occurrence table are ICD-10 codes.

For another one of the data quality checks 1202 in FIG. 12, management server 120 may perform a credible or plausible value check 1226 (see optional step 1126 in FIG. 11B). In a plausible value check 1226, validation unit 406 evaluates or determines whether values 705 of the EHR data 116 are credible based on the data quality compliance criteria 622. For example, validation unit 406 may parse the values 705 having a “date” to determine whether the dates are credible (e.g., start or death dates are not in the future, end dates do not occur before start dates, start dates are greater than a minimum date, such as “1900-01-01”, etc.). In another example, certain values 705 may not be set to NULL based on the data quality compliance criteria 622. Thus, validation unit 406 may parse one or more fields 704 to determine whether the fields 704 are populated with NULL values or non-NULL values. In another example, data quality compliance criteria 622 may comprise expected ranges for values 705, statistical ranges for values 705, etc., for one or more fields 704. The expected ranges and/or statistical ranges may be based on historical data for a health partner 602-603, based on averaged data for a health partner 602-603 or multiple health partners 602-603, etc. Validation unit 406 may compare a value 705 to an expected range and/or a statistical range for validation.

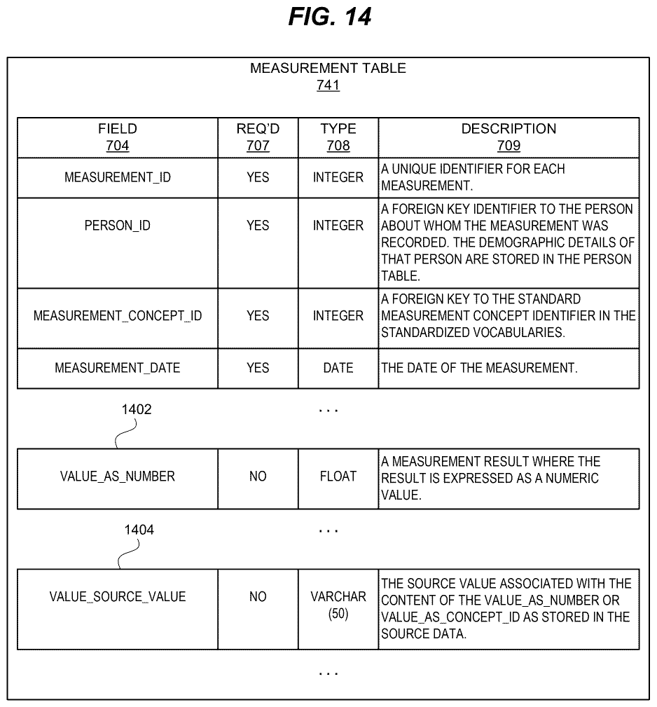

For another one of the data quality checks 1202 in FIG. 12, management server 120 may perform a truncated value check 1228 (see optional step 1128 in FIG. 11B). In EHR data 116, some values 705 may come across as a truncated value, such as “2” instead of “2.67”. In the truncated value check 1228, validation unit 406 may parse one or more values 705 to determine whether the values 705 have been incorrectly truncated. In an example, historical data may indicate certain values 705 that are susceptible to being truncated, and parse those values 705. As an example, values 705 contained in the measurement table 741 may be susceptible to being truncated. FIG. 14 illustrates a measurement table 741 in an illustrative embodiment. Some example fields 704 are illustrated for the measurement table 741 in FIG. 14. In particular, the measurement table 741 comprises a value_as_number field 1402 and a value_source_value field 1404. Validation unit 406 may compare the value_source_value field 1404 with the value_as_number field 1402 to determine whether the value 705 of the value_as_number field 1402 is inappropriately truncated.

For another one of the data quality checks 1202 in FIG. 12, management server 120 may perform a dropped condition or dropped diagnosis check 1230 (see optional step 1130 in FIG. 11B). In a dropped diagnosis check 1230, validation unit 406 determines or evaluates whether diagnoses that were documented in a previous submission (i.e., batch 610) from the health partner 602-603 have not been dropped from the current submission. For example, validation unit 406 may parse the EHR data 116 (e.g., the condition_occurrence table 740 or another table) of a batch 610 to identify conditions and/or diagnoses (e.g., diagnostic/medical/clinical codes recorded in the source data), and store a list of the conditions and/or diagnoses (e.g., a list of clinical codes). When a new batch 610 of EHR data 116 is received, validation unit 406 may parse the EHR data 116 (e.g., condition_occurrence table 740 or another table) of the new batch 610 to identify conditions and/or diagnoses, and determine that each of the conditions and/or diagnoses documented in a previous batch 610 are presented or included in the current batch 610.

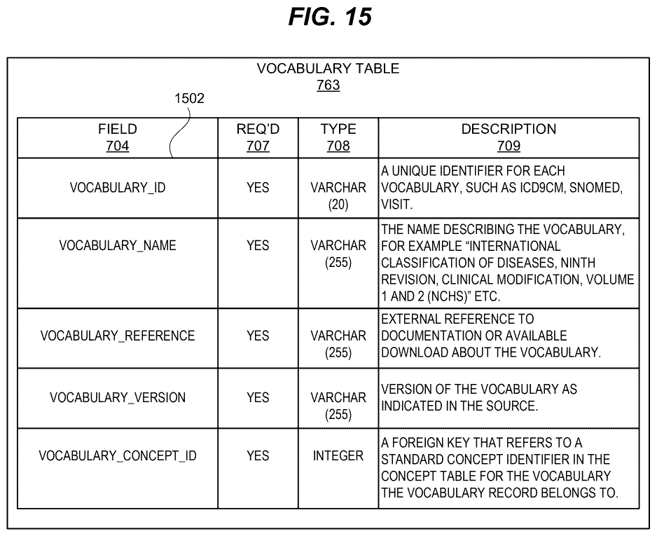

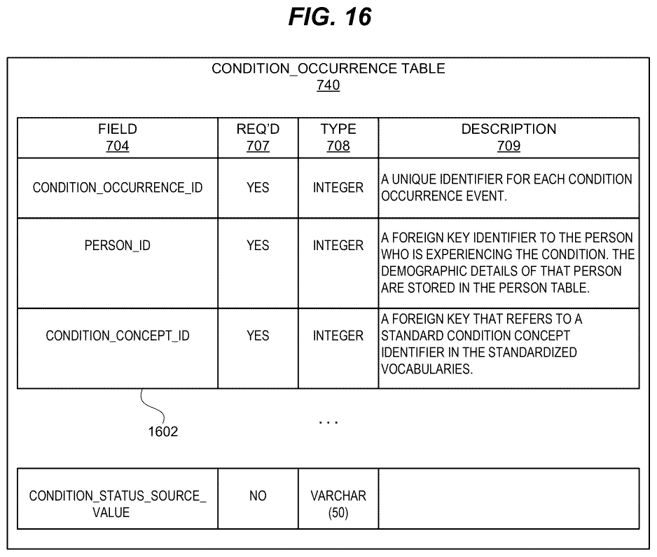

For another one of the data quality checks 1202 in FIG. 12, management server 120 may perform a vocabulary check 1232 (see optional step 1132 in FIG. 11B). In a vocabulary check 1232, validation unit 406 determines or evaluates whether a vocabulary(ies) used in the EHR data 116 is consistent or expected. For example, validation unit 406 may query the vocabulary table 763 of the EHR data 116, and compare a vocabulary ID in the vocabulary table 763 with a vocabulary ID in other tables. FIG. 15 illustrates the vocabulary table 763 and FIG. 16 illustrates a condition occurrence table (e.g., condition_occurrence table 740) in illustrative embodiments. Validation unit 406 may query the vocabulary table 763 to determine or identify a vocabulary ID 1502 (e.g., vocabulary_id) as shown in FIG. 15, such as for ICD, SNOMED, etc., and compare the vocabulary ID 1502 in the vocabulary table 763 with a condition concept ID 1602 (e.g., condition_concept_id) in the condition occurrence table as shown in FIG. 16. However, other vocabulary checks 1232 may be performed.

For another one of the data quality checks 1202 in FIG. 12, management server 120 may perform an age check 1234 (see optional step 1134 in FIG. 11B). In the age check 1234, validation unit 406 determines or confirms whether persons referenced in the EHR data 116 of the batch 610 are eighteen years or older. The age check 1234 may be specific to a consented dataset 614, where sequencing participants 206 may need to be eighteen years or older to consent to genetic sequencing.

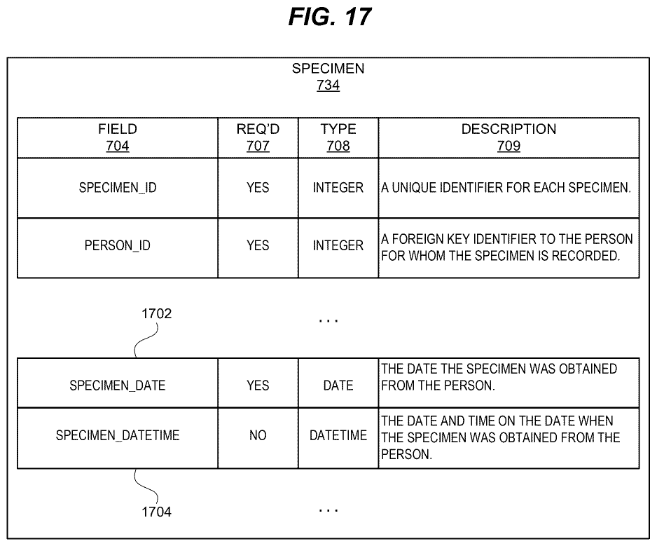

For another one of the data quality checks 1202 in FIG. 12, management server 120 may perform a transfusion check 1236 (see optional step 1136 in FIG. 11B). In the transfusion check 1236, validation unit 406 determines whether a biological sample 204 (e.g., blood, saliva, etc.) or specimen of a person was collected within thirty days of a blood transfusion for that person. The transfusion check 1236 is specific to a consented dataset 614, where a sequencing participant 206 submitted a biological sample 204 for genetic sequencing. For example, validation unit 406 may query the specimen table 734 of the EHR data 116 to identify a date of a biological sample 204 for a person (associated with a person ID), and determine whether the biological sample 204 was collected within thirty days of a blood transfusion for that person. FIG. 17 illustrates the specimen table 734 in an illustrative embodiment. Validation unit 406 may parse the specimen date 1702 (e.g., specimen_date) and/or the specimen datetime 1704 (e.g., specimen_datetime) of the specimen table 734 to identify a date of a biological sample 204 for a person, and determine whether the biological sample 204 was collected within thirty days of a blood transfusion.

For another one of the data quality checks 1202 in FIG. 12, management server 120 may perform a core lab percentage check 1238 (see optional step 1138 in FIG. 11B). In the core lab percentage check 1238, validation unit 406 determines whether a threshold percentage of core laboratory results is present in the measurement table 741.

For another one of the data quality checks 1202 in FIG. 12, management server 120 may perform a dropped genetic testing check 1240 (see optional step 1140 in FIG. 11B). In a dropped genetic testing check 1240, validation unit 406 determines or evaluates whether genetic testing IDs (e.g., genetic test results, order IDs, kit IDs, etc.) that were documented in a previous submission (i.e., batch 610) have not been dropped from the current submission. For example, validation unit 406 may parse the EHR data 116 (e.g., measurement table 741, specimen table 734, or another table) of a batch 610 to identify genetic testing IDs (e.g., genetic test results, order IDs, kit IDs, etc.), and store a list of genetic testing IDs. When a new batch 610 of EHR data 116 is received, validation unit 406 may parse the EHR data 116 (e.g., measurement table 741, specimen table 734, or another table) of the new batch 610 to identify genetic testing IDs, and determine that each of the genetic testing IDs documented in a previous batch 610 are presented or included in the current batch 610.

As described in FIG. 8, validation unit 406 may be implemented in the AWS Glue service 850, where AWS Glue Data Quality 852 evaluates objects (e.g., EHR data 116) stored in the AWS Glue Data Catalog, and performs or enforces data quality checks on the objects. The DQDL rule set 854 may set out the rules used to evaluate EHR data 116 for data quality.

In FIG. 11A, management server 120 determines whether the structurally-compliant EHR data 116 of the batch 610 is in compliance with the data quality compliance criteria 622 according to or based on the data quality validation 1200 (step 1104). For example, data management controller 404 may output a control signal 405 to validation unit 406 to determine whether the EHR data 116 of the batch 610 is in compliance with the data quality compliance criteria 622. Management server 120 may detect or identify any errors or issues (i.e., non-compliant data) in the EHR data 116, and determine whether the errors or issues exceed compliance thresholds 626. For any errors or issues detected in the EHR data 116, management server 120 may determine whether the EHR data 116 passes the data quality validation 1200 based on the compliance thresholds 626. For example, certain errors may not have a significant impact on the utilization of the EHR data 116 (e.g., low priority errors), while other errors (e.g., critical or moderate errors) may have an impact on the utilization of the EHR data 116. The compliance thresholds 626 may therefore define or specify which errors are acceptable and which errors are not.

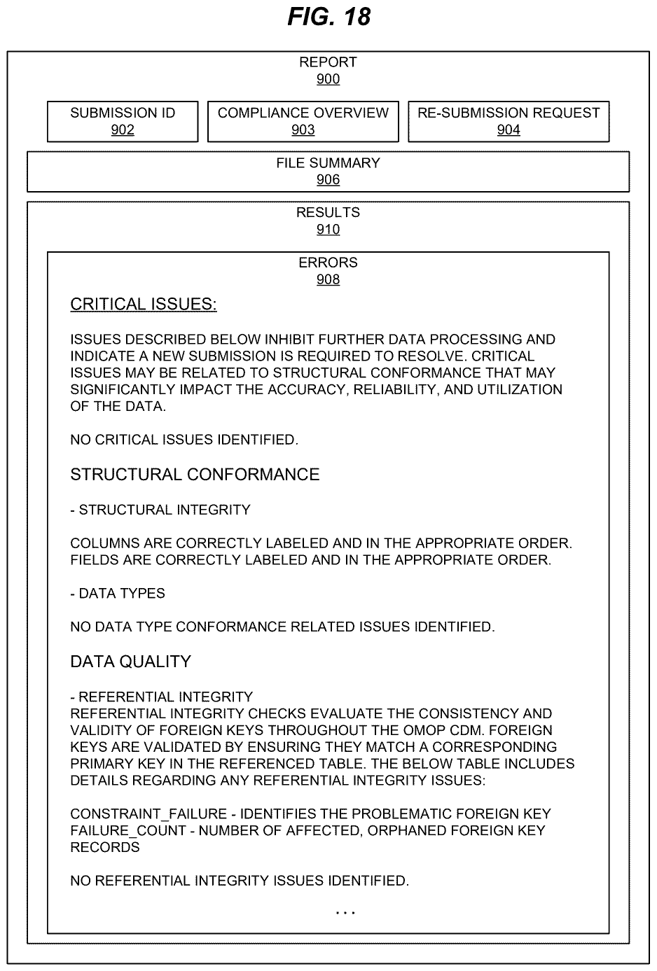

In an embodiment, management server 120 may generate a report 900 indicating results of the data quality validation 1200 (optional step 1116 in FIG. 11A). For example, data management controller 404 may output a control signal 405 to validation unit 406 to generate the report 900. FIG. 18 illustrates a report 900 in an illustrative embodiment. The report 900 comprises information, statistics, etc., regarding differences, errors, or issues detected in the EHR data 116 based on the data quality validation 1200. The report 900 of data quality validation 1200 may be combined with any prior reports of the batch 610 generated for structural conformance validation 800, or may be separate. The report 900 may include a submission ID 902 indicating the batch 610 of EHR data 116 submitted by a health partner 602-603, an overview 903 highlighting any errors or issues encountered while processing the batch 610 of EHR data 116 indicated by the submission ID 902, a re-submission request 904 for a health partner 602-603 to re-submit a corrected dataset, such as when critical errors have been identified, and a file summary 906. The report 900 includes results 910 of the data quality validation 1200. The results 910 may include or indicate one or more errors 908 detected in the EHR data 116, may indicate a priority 624 of the error(s) 908, and/or other information. An example of the results 910 is provided in FIG. 18 merely as an example, and the content of the results 910 may vary as desired.

In an embodiment, management server 120 may correct one or more errors 908 (e.g., non-compliant data) detected in the EHR data 116 (optional step 1118 in FIG. 11A), such as based on the priority 624 of the errors 908. According to the compliance criteria 620, certain errors 908 may be automatically corrected by the management server 120, such as low priority errors. For example, a death date incorrectly indicating a future date may be modified to the correct date. Further, management server 120 may seek feedback from a human, a domain expert, or the like to correct certain errors.

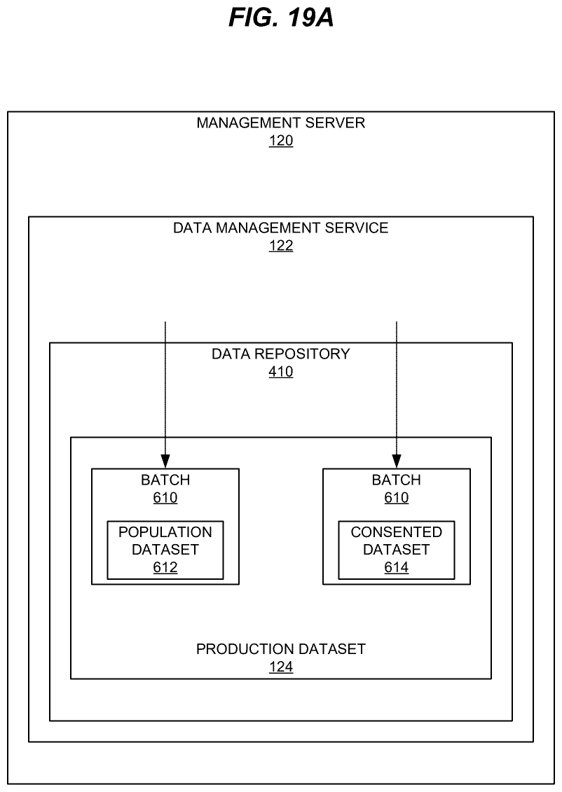

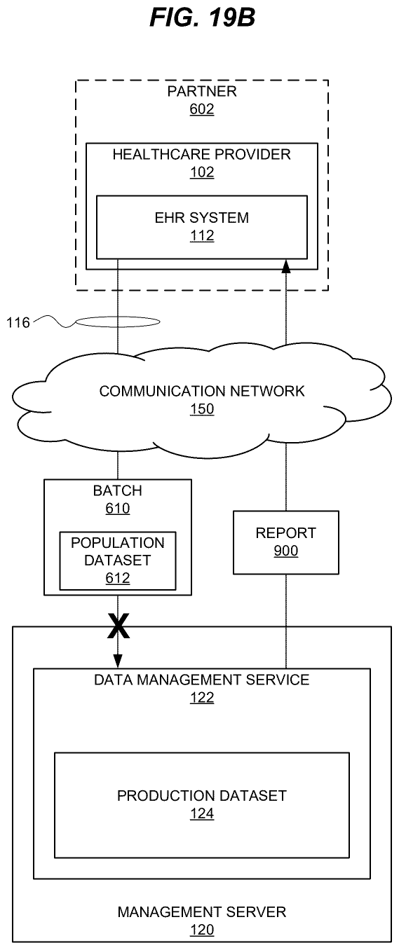

Based on the determination in step 1104, management server 120 accepts the structurally-compliant EHR data 116 of the batch 610 from a health partner 602-603 or rejects the EHR data 116. More particularly, when the structurally-compliant EHR data 116 of the batch 610 is compliant based on the data quality validation 1200 (i.e., in compliance with the data quality compliance criteria 622), management server 120 accepts the structurally-compliant EHR data 116 as compliant EHR data 116 (step 1106), and stores the compliant EHR data 116 in the production dataset 124 (step 1108) or otherwise merges the compliant EHR data 116 into the production dataset 124. For example, data management controller 404 may output a control signal 405 to validation unit 406 to store the compliant EHR data 116 in data repository 410 as part of the production dataset 124. FIG. 19A illustrates accepting of compliant EHR data 116 in the production dataset 124 in an illustrative embodiment. In this example, the compliant EHR data 116 of the batch 610 is compliant based on both structural conformance validation 800 and data quality validation 1200. Thus, management server 120 stores the compliant EHR data 116 of the batch 610 in the production dataset 124. The compliant EHR data 116 of the batch 610 will be available for further research, analysis, or another type of post-processing. One technical benefit is management server 120 verifies the quality of the EHR data 116 received from a health partner 602-603 before addition to the production dataset 124 in order to assemble a robust production dataset 124. Management server 120 may then send a report 900 to the health partner 602-603 (step 1110). For example, data management controller 404 may output a control signal 405 to network interface component 402 to send the report 900 to the health partner 602-603. Because the EHR data 116 of the batch 610 is compliant based on the structural conformance validation 800 and the data quality validation 1200, the report 900 will likely not be accompanied by a re-submission request 904.

When structurally-compliant EHR data 116 of a batch 610 is non-compliant based on the data quality validation 1200 (i.e., not in compliance with the data quality compliance criteria 622), management server 120 rejects the structurally-compliant EHR data 116 as non-compliant EHR data 116 (step 1112), and sends a report 900 to the health partner 602-603 (step 1114). In an embodiment, management server 120 may send a re-submission request 904 to the health partner 602-603 in the report 900, separate from the report 900, etc., to submit a modified batch 610 of EHR data 116. In another embodiment, management server 120 may wait for the next submission from the health partner 602-603 that is modified based on the report 900. FIG. 19B illustrates rejecting of the structurally-compliant EHR data 116 from the production dataset 124 in an illustrative embodiment. In this example, the structurally-compliant EHR data 116 of the batch 610 is not compliant based on the data quality validation 1200. Thus, management server 120 rejects the structurally-compliant EHR data 116 (i.e., does not store the structurally-compliant EHR data 116 of the batch 610 in the production dataset 124), and sends the report 900 to the health partner 602-603. One technical benefit is management server 120 reports non-compliant EHR data 116 to a health partner 602-603 to allow the heath partner 602-603 to submit a corrected dataset, which improves the quality of the production dataset 124 that is compiled from EHR data 116.

In storing the compliant EHR data 116 of the batch 610 in the production dataset 124, management server 120 may further process the compliant EHR data 116 as described below. In FIG. 11C, management server 120 may transform the compliant EHR data 116 to a service-specific format (step 1152), such as a service-specific OMOP CDM format. For example, data management controller 404 may output a control signal 405 to preparation unit 408 to transform the compliant EHR data 116. The service-specific format may vary slightly from the data model 801, and includes additional information desired by the data management service 122. For example, preparation unit 408 may add a table 702 or field 704 with health partner information (optional step 1160), such as a health partner ID. In another example, preparation unit 408 may standardize units indicated in the compliant EHR data 116 (optional step 1162), such as grams to milligrams. Management server 120 may transform the compliant EHR data 116 in other ways as desired before addition to the production dataset 124.

Management server 120 may annotate the compliant EHR data 116 (step 1154). For example, data management controller 404 may output a control signal 405 to preparation unit 408 to annotate the compliant EHR data 116.

Management server 120 may then merge or otherwise store the compliant EHR data 116 in the production dataset 124 (step 1156). For example, data management controller 404 may output a control signal 405 to validation unit 406 or preparation unit 408 to load the compliant EHR data 116 to a storage location of the production dataset 124. One technical benefit is the compliant EHR data 116 supplements the production dataset 124, which may be used for further analysis.

Management server 120 may send a report 900 to the health partner 602-603 (step 1158) indicating the results of the structural conformance validation 800 and the data quality validation 1200.

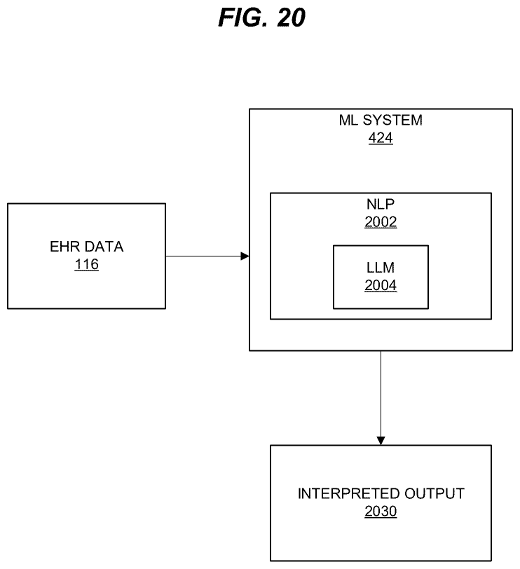

In an embodiment, management server 120 may implement a ML system 424 to process the EHR data 116 as described above. FIG. 20 is a diagram illustrating use of an ML system 424 to process the EHR data 116 in an illustrative embodiment. ML system 424 may be trained to interpret human language. Some examples of ML system 424 are a Natural Language Processing (NLP) model 2002, a Large Language Model (LLM) 2004, etc. Thus, ML system 424 may be implemented to interpret the EHR data 116, and generate interpreted output 2030 indicating one or more errors 908 detected in the EHR data 116.

### Example

In the following example, additional processes, systems, and methods may be described in the context of data management. The processes, systems, and methods described in this example may be incorporated in embodiments described above as desired.

In this example, it is assumed that a health partner 603 submits a batch of EHR data 116 comprising a consented dataset 614 for a plurality of sequencing participants 206. In an ingestion phase 600, management server 120 receives the consented dataset 614 in OMOP CDM format 712. Management server 120 performs structural conformance validation 800 on the consented dataset 614 based on the structural compliance criteria 621 by comparing the incoming consented dataset 614 to a data model 801 defined in the structural compliance criteria 621. Assume, for this example, that a table 702 is missing in the consented dataset 614 when compared to the data model 801, and the missing table 702 is considered a critical error. Structural conformance validation 800 will therefore fail, and management server 120 rejects the consented dataset 614. Management server 120 sends a report 900 to the health partner 603 indicating the error and including a re-submission request 904.

Management server 120 then receives a re-submission from the health partner 603 comprising a modified consented dataset 614. Management server 120 performs structural conformance validation 800 on the consented dataset 614 as modified based on the structural compliance criteria 621 by comparing the consented dataset 614 to the data model 801. In this instance, structural conformance validation 800 passes, and management server 120 accepts the consented dataset 614. Management server 120 then stores the consented dataset 614 in data repository 410 for further validation.

After the ingestion phase 600, management server 120 performs data quality validation 1200 on the consented dataset 614. Assume, for this example, that the consented dataset 614 includes information on a sequencing participant 206 that is under the age of eighteen, and inclusion of this information is considered a critical error. Data quality validation 1200 will therefore fail, and management server 120 rejects the consented dataset 614. Management server 120 sends a report 900 to the health partner 603 indicating the error, and including a re-submission request 904. Management server 120 also deletes the consented dataset 614 from the data repository 410.

Management server 120 then receives a re-submission from the health partner 603 comprising a modified consented dataset 614. Management server 120 performs structural conformance validation 800 on the consented dataset 614 as modified based on the structural compliance criteria 621 by comparing the consented dataset 614 to the data model 801. In this instance, structural conformance validation 800 passes, and management server 120 accepts the consented dataset 614. Management server 120 then stores the consented dataset 614 in data repository 410. After the ingestion phase 600, management server 120 performs data quality validation 1200 on the consented dataset 614. In this instance, data quality validation 1200 passes, and management server 120 accepts the consented dataset 614. Management server 120 then stores the consented dataset 614 as part of the production dataset 124 (e.g., after any other processing, such as transforming, annotating, etc., as described above). Management server 120 also sends a report 900 to the health partner 603 indicating results of the validation. One technical benefit is the veracity of the production dataset 124 is improved by validating the consented dataset 614 that is added.

As described above, a consented dataset 614 includes or is linked to genetic information (e.g., sequencing data 142) for sequencing participants 206. In general, laboratory procedures related to genetics may include accessioning, sample plating, storage, extraction, library preparation, enrichment, and sequencing processes. These processes acquire genetic material from a sample, separate the genetic material from other constituents, duplicate the genetic material, and quantify the genetic material order to determine a swathe of sequence data, such as an exome or entire genome for a subject (e.g., a human, an animal, a pathogen, an organelle, etc.).

Sequencing may be performed according to any of a variety of techniques, including short-read and long-read techniques. In one embodiment, the sequencing is performed as Sequencing by Synthesis (SBS) at genetic analyzer equipment. For example, sets of enriched libraries of genetic material bound to probes in earlier steps may be transferred to a flow cell, and annealed to oligonucleotide probes within the flow cell. At this stage, the contents of multiple wells may be applied to the same flow cell, because the libraries within those wells are tagged with the chemical identifiers. In one embodiment, the chemical identifiers comprise nucleotide sequences that are detectable during the sequencing process to determine a corresponding Laboratory Sample Identifier (LSI).

Complementary sequences may then be created via enzymatic extension to create a double-stranded portion of genetic material. The double-stranded genetic material may then be denatured, and the library fragment may be washed away. Bridge amplification may then be performed to create copies of the remaining molecule in a localized cluster. For example, a cluster may comprise twenty to fifty copies of the same molecule, localized to a location the size smaller than a pinhead on the flow cell.

Sequencing primers are annealed to library adapters in order to prepare the flow cell for SBS. During SBS, the sequencing primer uses reverse terminator fluorescent oligonucleotides, one base per cycle, for a number of cycles (e.g., one hundred and fifty cycles) in the forward direction. After the addition of each nucleotide, clusters are excited by a light source, resulting in fluorescence which can be measured. The emission wavelength and signal intensity for each cluster determines a base call for that cluster. Fluorescent moieties are then flushed from the flow cell. A chemical group blocking a 3′ end of the fragment is then removed, enabling a subsequent nucleotide to be read. This tightly controls nucleotide addition and detection.

Base calls across cycles at the same physical location on the flow cell occur at the same cluster, and hence indicate sequential reads for copies of the same fragment of the genetic material. After each cycle, denaturing and annealing are performed to extend the index primer. A complementary reverse strand is created and extended via bridge amplification. The reverse strand is then read in the reverse direction for a number of cycles, in a manner similar to reads in the forward direction.

Depending on whether a complete human genome, or another set of genomic data, is being tested, different reagents (e.g., probes, primers, etc.) may be chosen. That is, different reagents may be utilized for library preparation for a pathogen (e.g., bacteria, virus) or an organelle (e.g., mitochondria) than for a human genome. Pathogens exhibiting Ribonucleic Acid (RNA) genomes may have their genetic material translated to DNA before sequencing, enrichment, and/or library preparation are performed, via known techniques, such as Next Generation Sequencing (NGS) techniques.

Throughout the processes discussed above, the laboratory environment may be carefully controlled to ensure quality. For example, temperature within each segment of the laboratory may be carefully monitored and controlled, and ultraviolet lighting or other features capable of inactivating genetic material may be carefully positioned to ensure that contamination does not occur.

In some embodiments, genetic material is used for detection of a pathogen rather than for sequencing. Detecting a pathogen may involve the use of a real-time Polymerase Chain Reaction (PCR) system that performs PCR. The real-time PCR system may further add a reactive agent to individual wells of a library preparation microplate, that fluoresces when bound to genetic material for the pathogen. By analyzing fluorescence at known periods of time after PCR has initiated, presence of a pathogen is determined. Genetic testing for a pathogen may thereby forego sequencing in some embodiments.

Raw sequence data generated during synthesis may be stored in a file format, such as Binary Base Call (BCL), depending on the sequencing equipment used. This raw data may be fed to an analytical pipeline, such as a cloud-based computing environment. Raw sequence data may be processed by the analytical pipeline into a second format, such as a text-based FASTQ format, that reports the sequence information (i.e., the sequence reads) and corresponding quality scores. The second format is then analyzed to perform alignment of sequence reads to a reference genome, such as a reference genome reported in a Browser Extensible Data (BED) file. The aligned sequence data may be reported as a Binary Alignment Map (BAM) file. The aligned sequence data may then be called, resulting in a Variant Call Format (VCF) file reporting called variants at each location of the genome that was sequenced, together with secondary metrics, such as quality indicator metrics.

The called sequence data may be provided to a data analyst via a User Interface (UI), such as a GUI presented via a display. The technician may then validate the resulting called sequence data and release it for reporting to subjects, healthcare providers, and/or scientists.

Although specific embodiments were described herein, the scope of the invention is not limited to those specific embodiments. The scope of the invention is defined by the following claims and any equivalents thereof.

What is claimed is:

1. An apparatus, comprising:
a network interface configured to communicate over a communication network; and
a processor and memory, wherein
the memory is configured to store structural compliance criteria and data quality compliance criteria; and
the processor is configured to execute an algorithm to:
during an ingestion phase:
receive a batch of Electronic Health Record (EHR) data from a health partner via the network interface;
perform structural conformance validation of the EHR data based on the structural compliance criteria by comparing the EHR data to a data model defined in the structural compliance criteria;
determine whether the EHR data is in compliance with the structural compliance criteria according to the structural conformance validation;
reject the EHR data when not in compliance with the structural compliance criteria; and
accept the EHR data as structurally-compliant EHR data when in compliance with the structural compliance criteria;
after the ingestion phase,
perform data quality validation of the structurally-compliant EHR data based on the data quality compliance criteria;
determine whether the structurally-compliant EHR data is in compliance with the data quality compliance criteria according to the data quality validation;
reject the structurally-compliant EHR data when not in compliance with the data quality compliance criteria;
accept the structurally-compliant EHR data as compliant EHR data when in compliance with the data quality compliance criteria; and
store the compliant EHR data in a production dataset.

2. The apparatus of claim 1, wherein the processor is further configured to execute the algorithm to:
send a report to the health partner via the network interface, when the EHR data is not in compliance with the structural compliance criteria, including:
results of the structural conformance validation indicating one or more errors detected in the EHR data; and
a re-submission request to re-submit the EHR data.

3. The apparatus of claim 1, wherein:
the structural conformance validation comprises a suite of structural conformance checks including one or more of:
a table check to determine whether tables of the EHR data are in conformance with the data model;
a field check to determine whether fields of the EHR data are in conformance with the data model; and
a data type check to validate data types for values provisioned in the EHR data based on the structural compliance criteria.

4. The apparatus of claim 1, wherein the processor is further configured to execute the algorithm to:
send a report to the health partner via the network interface, when the structurally-compliant EHR data is not in compliance with the data quality compliance criteria, including:
results of the data quality validation indicating one or more errors detected in the structurally-compliant EHR data; and
a re-submission request to re-submit the EHR data.

5. The apparatus of claim 1, wherein:
the data quality validation comprises a suite of data quality checks including one or more of:
a referential integrity check to evaluate whether relationships between fields of the structurally-compliant EHR data are valid;
a person identifier mismatch check to determine whether, for each table of the structurally-compliant EHR data provisioned with a person identifier and a visit identifier, maps the visit identifier to a same person identifier; and
an International Classification of Diseases (ICD) switch check to determine whether codes used in the structurally-compliant EHR data are ICD-10 codes.

6. The apparatus of claim 5, wherein:
the suite of data quality checks further includes one or more of:
a plausible value check to determine whether values of the structurally-compliant EHR data are credible based on the data quality compliance criteria;
a truncated value check to parse one or more of the values of the structurally-compliant EHR data to determine whether the values have been incorrectly truncated;
a dropped diagnosis check to determine whether diagnoses that were documented in a previous batch from the health partner have not been dropped from the received batch; and
a vocabulary check to determine whether a vocabulary used in the structurally-compliant EHR data is consistent by querying a vocabulary table of the structurally-compliant EHR data, and comparing a vocabulary identifier in the vocabulary table with the vocabulary identifier in other tables.

7. The apparatus of claim 6, wherein:
the suite of data quality checks further includes one or more of:
an age check to determine whether persons referenced in the structurally-compliant EHR data are eighteen years or older;
a transfusion check to determine whether a biological sample of a person submitted for genetic sequencing was collected within thirty days of a blood transfusion for the person; and
a dropped genetic testing check to determine whether genetic testing identifiers that were documented in a previous batch from the health partner have not been dropped from the received batch.

8. The apparatus of claim 1, wherein the processor is further configured to execute the algorithm to:
transform the compliant EHR data to a service-specific format that adds a table or field to the compliant EHR data with a health partner identifier for the health partner;
annotate the compliant EHR data; and
merge the compliant EHR data into the production dataset.

9. The apparatus of claim 1, wherein:
the EHR data is received in Observational Medical Outcomes Partnership (OMOP) Common Data Model (CDM) format.

10. A method, comprising:
during an ingestion phase:
receiving a batch of Electronic Health Record (EHR) data from a health partner via a communication network;
performing structural conformance validation of the EHR data based on structural compliance criteria stored in memory by comparing the EHR data to a data model defined in the structural compliance criteria;
determining whether the EHR data is in compliance with the structural compliance criteria according to the structural conformance validation;
rejecting the EHR data when not in compliance with the structural compliance criteria; and
accepting the EHR data as structurally-compliant EHR data when in compliance with the structural compliance criteria;
after the ingestion phase,
performing data quality validation of the structurally-compliant EHR data based on data quality compliance criteria stored in memory;
determining whether the structurally-compliant EHR data is in compliance with the data quality compliance criteria according to the data quality validation;
rejecting the structurally-compliant EHR data when not in compliance with the data quality compliance criteria;
accepting the structurally-compliant EHR data as compliant EHR data when in compliance with the data quality compliance criteria; and
storing the compliant EHR data in a production dataset.

11. The method of claim 10, further comprising:
sending a report to the health partner via the communication network, when the EHR data is not in compliance with the structural compliance criteria, including:
results of the structural conformance validation indicating one or more errors detected in the EHR data; and
a re-submission request to re-submit the EHR data.

12. The method of claim 10, wherein:
the structural conformance validation comprises a suite of structural conformance checks including one or more of:
a table check to determine whether tables of the EHR data are in conformance with the data model;
a field check to determine whether fields of the EHR data are in conformance with the data model; and
a data type check to validate data types for values provisioned in the EHR data based on the structural compliance criteria.

13. The method of claim 10, further comprising:
sending a report to the health partner via the communication network, when the structurally-compliant EHR data is not in compliance with the data quality compliance criteria, including:
results of the data quality validation indicating one or more errors detected in the structurally-compliant EHR data; and
a re-submission request to re-submit the EHR data.

14. The method of claim 10, wherein:
the data quality validation comprises a suite of data quality checks including one or more of:
a referential integrity check to evaluate whether relationships between fields of the structurally-compliant EHR data are valid;
a person identifier mismatch check to determine whether, for each table of the structurally-compliant EHR data provisioned with a person identifier and a visit identifier, maps the visit identifier to a same person identifier; and
an International Classification of Diseases (ICD) switch check to determine whether codes used in the structurally-compliant EHR data are ICD-10 codes.

15. The method of claim 14, wherein:
the suite of data quality checks further includes one or more of:
a plausible value check to determine whether values of the structurally-compliant EHR data are credible based on the data quality compliance criteria;
a truncated value check to parse one or more of the values of the structurally-compliant EHR data to determine whether the values have been incorrectly truncated;
a dropped diagnosis check to determine whether diagnoses that were documented in a previous batch from the health partner have not been dropped from the received batch; and
a vocabulary check to determine whether a vocabulary used in the structurally-compliant EHR data is consistent by querying a vocabulary table of the structurally-compliant EHR data, and comparing a vocabulary identifier in the vocabulary table with the vocabulary identifier in other tables.

16. The method of claim 15, wherein:
the suite of data quality checks further includes one or more of:
an age check to determine whether persons referenced in the structurally-compliant EHR data are eighteen years or older;
a transfusion check to determine whether a biological sample of a person submitted for genetic sequencing was collected within thirty days of a blood transfusion for the person; and
a dropped genetic testing check to determine whether genetic testing identifiers that were documented in a previous batch from the health partner have not been dropped from the received batch.

17. The method of claim 10, further comprising:
transforming the compliant EHR data to a service-specific format that adds a table or field to the compliant EHR data with a health partner identifier for the health partner;
annotating the compliant EHR data; and
merging the compliant EHR data into the production dataset.

18. A non-transitory computer readable medium embodying programmed instructions executed by a processor, wherein the instructions direct the processor to implement a method comprising:
during an ingestion phase:
receiving a batch of Electronic Health Record (EHR) data from a health partner via a communication network;
performing structural conformance validation of the EHR data based on structural compliance criteria stored in memory by comparing the EHR data to a data model defined in the structural compliance criteria;
determining whether the EHR data is in compliance with the structural compliance criteria according to the structural conformance validation;
rejecting the EHR data when not in compliance with the structural compliance criteria; and
accepting the EHR data as structurally-compliant EHR data when in compliance with the structural compliance criteria;
after the ingestion phase,
performing data quality validation of the structurally-compliant EHR data based on data quality compliance criteria stored in memory;
determining whether the structurally-compliant EHR data is in compliance with the data quality compliance criteria according to the data quality validation;
rejecting the structurally-compliant EHR data when not in compliance with the data quality compliance criteria;
accepting the structurally-compliant EHR data as compliant EHR data when in compliance with the data quality compliance criteria; and
storing the compliant EHR data in a production dataset.

19. The computer readable medium of claim 18, wherein the method further comprises:
sending a report to the health partner via the communication network, when the EHR data is not in compliance with the structural compliance criteria, including:
results of the structural conformance validation indicating one or more errors detected in the EHR data; and
a re-submission request to re-submit the EHR data.

20. The computer readable medium of claim 18, wherein the method further comprises:
sending a report to the health partner via the communication network, when the structurally-compliant EHR data is not in compliance with the data quality compliance criteria, including:
results of the data quality validation indicating one or more errors detected in the structurally-compliant EHR data; and
a re-submission request to re-submit the EHR data.
